(19) United States
(12) Patent Application Publication
(10) Pub. No.: US 2025/0360192 A1
(43) Pub. Date: Nov. 27, 2025

(54) STAPHYLOCOCCUS AUREUS VACCINE

(71) Applicant: THE REGENTS OF THE UNIVERSITY OF CALIFORNIA, Oakland, CA (US)

(72) Inventors: George Yen-Hsi LIU, La Jolla, CA (US); Chih-Ming TSAI, La Jolla, CA (US)

(21) Appl. No.: 18/872,768

(22) PCT Filed: Jun. 8, 2023
(86) PCT No.: PCT/US2023/068158

§ 371 Date: Dec. 6, 2024

Related U.S. Application Data
(60) Provisional application No. 63/350,385, filed on Jun. 8, 2022.

(51) Int. Cl. A61K 39/085; A61K 39/00; A61P 31/04

(52) U.S. Cl. CPC A61K 39/085; A61P 31/04; A61K 2039/545; A61K 2039/575

(57) ABSTRACT

A vaccine for Staphylococcus aureus is disclosed. A method for producing a vaccine for S. aureus is also disclosed. A method for immunizing a human against S. aureus infection is further disclosed.

### CROSS-REFERENCE TO RELATED APPLICATIONS

This application claims priority from U.S. Provisional Application Ser. No. 63/350,385 filed on Jun. 8, 2022, which is incorporated herein by reference in its entirety.

### GOVERNMENT RIGHTS

This invention was made with government support under R01AI144694 awarded by the National Institutes of Health (NIH). The government has certain rights in the invention.

### SEQUENCE LISTING

The Sequence Listing, which is a part of the present disclosure, includes a computer readable form and a written sequence listing comprising nucleotide and/or amino acid sequences of the present invention. The sequence listing information recorded in computer readable form is identical to the written sequence listing. The subject matter of the Sequence Listing is incorporated herein by reference in its entirety.

### FIELD

The present teachings relate to a novel Staphylococcus aureus vaccine and methods of using and producing same.

### INTRODUCTION

Staphylococcus aureus (“SA” or “S. aureus”) is a major cause of health burden and has been the target of vaccine development for over a century. Although many successful candidates have emerged from laboratory animal studies, all passive and active vaccines taken to clinical trials have failed in humans without a clear explanation. Whereas mice in a laboratory setting have infrequent exposure to SA, human infants have a colonization rate of over 50% during the first two months of life. As a pathobiont, SA elicits abundant antibodies (Ab) against SA in most subjects. However, these anti-SA Ab are not effective against SA infection as individuals with B cell deficiency are not more susceptible to SA infections than normal subjects. SA has developed many mechanisms to evade the host adaptive immune system. Thus, it was considered if pre-existing immune response to SA can play a role in SA vaccine failures.

To address this question, the most notable SA vaccine “failures” to date was reexamined. That study targeted the iron-regulated surface determinant protein B (IsdB), a critical antigen for acquisition of host iron. Although the vaccine induced robust titers of IsdB Ab in subjects, the vaccine was ineffective in the Phase 3 clinical trial.

Therefore, to address the ineffectiveness of current models of immunizing against SA infection, a new vaccine strategy is needed.

### SUMMARY

A first aspect of the present teachings is directed to a vaccine composition. The vaccine composition comprises a recombinant NEAT2 polypeptide, or variant or fragment thereof, comprising an amino acid sequence corresponding to SEQ ID NO: 1. Another aspect of the present teachings is directed to a polynucleotide vaccine composition comprising a polynucleotide sequence corresponding to SEQ ID NO: 2, or variant or fragment thereof.

Another aspect of the present teachings is directed to a recombinant NEAT2 polypeptide, or variant or fragment thereof, having immunogenic activity.

Other aspects of the present teachings are directed to methods of treating a staphylococcal infection in a subject using the vaccine compositions described herein.

Yet another aspect of the present teachings includes the enhancement of the immunogenicity of a previously unprotective vaccine by the administration of the novel recombinant NEAT2 polypeptide to a subject in need thereof.

These and other features, aspects and advantages of the present teachings will become better understood with reference to the following description, examples and appended claims.

### DESCRIPTION OF THE DRAWINGS

Those of skill in the art will understand that the drawings, described below, are for illustrative purposes only. The drawings are not intended to limit the scope of the present teachings in any way.

FIGS. 1a-1h: IsdB immunization is not protective in mice previously infected with S. aureus. 1a, Experimental setting. 1b, Serum anti-IsdB IgG after 1-3 SA (LAC) infections (n=15 per condition). 1c-1d, Tissue bacterial burden after 1, 2 (c), or 3 (d) LAC infections, then immunizations and SA challenge as per FIG. 1A (n=5-10 per condition). 1e, Kaplan-Meyer plot of mice treated as in FIG. 1A (3 LAC infections plus vaccines) with a final SA challenge with an LD₉₀ dose (n=10 per condition). N/IsdB: Naïve mice vaccinated with IsdB, N/alum: naïve mice given adjuvant alone; LAC/IsdB: LAC-infected mice vaccinated with IsdB; LAC/alum: LAC-infected mice given adjuvant alone (LAC/alum). 1f, Antigen-specificity of vaccine suppression: Mice were infected with WT or DIsdB/HarA SA Becker, immunized, then challenged with WT Becker (n=10 per condition). 1g-1h, Effect of prior SA infection on humoral protection conferred by IsdB vaccine. Naïve mice received i.v. splenic B cells (g) or total IgG (h) from donor mice treated per FIG. 1a, then received SA i.p. 24 h later (n=8-10 per condition). Each data point represents one mouse; bars denote median and dashed lines indicate the limit of detection (1c, 1d and 1f to 1h). One-way ANOVA with Kruskal-Wallis test (b to d and f to h) or log rank test (e).

FIGS. 2a-2j: IsdB vaccination recalls a non-protective IsdB-specific response from prior S. aureus infection. 2a, Serum IsdB-specific IgG 7d after IsdB immunization with or without prior infection per FIG. 1a (n=5 per condition). 2b, Avidity of IsdB-specific Ab measured in the presence of increasing urea concentrations (n=2). 2c, Opsonophagocytic killing (OPK) of SA (LAC) by primary mouse neutrophils in the presence of immunized sera (n=3). 2d, Ribbon representation of interaction between immunized mouse sera (from FIG. 1a) and overlapping IsdB peptide library. Darker shades indicate Intensity of Ab binding to specific domains: light grey (0 or low intensity) to dark grey (over 1000). Arrow: heme-binding motif. 2e, Heme-dependent growth of SA in the presence of immunized mouse sera (n=3). 2f, Heme-dependent growth of SA in the presence of mAbs from clones derived from a IsdB-specific B cell hybridoma library, generated by IsdB vaccination of naïve mice (n=3). 2g, Adoptive transfer of IsdB-specific mAbs (FIG. 2f, 0.5 mg/kg) prevents SA infection (n=8-11 per condition). 2h, Alluvial plot showing the top 40 of clonotypes from LAC, LAC/IsdB and N/IsdB. 2i, Rapid rise of IsdB-specific antibody titer 7d after first the IsdB vaccination in SA pre-infected mice (n=10 per condition). 2j, ELISpot assay of IsdB-specific IgG-secreting splenic cells (ASCs) 7d after the first IsdB vaccination. Bar represents group median. Each data point represents one mouse; bars denote median and dashed lines indicate the limit of detection. Error bars represent means±SD. Student's t test (a to c, e and f) or one-way ANOVA with Kruskal-Wallis test (2g, 2i and 2j).

FIGS. 3a-3g: Competition for IsdB between non-protective and protective antibodies determines outcome of S. aureus infection. 3a, Anti-SA immunity in mice co-injected with equal volumes (50 μl i.p.) of protective (N/IsdB) and non-protective (LAC/IsdB) IsdB-specific sera (n=8 per condition). 3b, Anti-SA immunity in mice co-injected with equal amounts (25 μg i.p.) of protective (N/IsdB) and non-protective (LAC/IsdB) IsdB-specific Ab (n=5-8 per condition). 3c, Effect of IsdB-specific Ab competition on mouse neutrophil OPK (n=3). 3d, IsdB binding (ELISA) by protective IsdB-specific Ab (N/IsdB, 1 μg/ml) in the presence of non-protective IsdB-specific Ab (LAC or LAC/IsdB). 3e, Schematic of the IsdB, N1 and N2 proteins used for vaccination. Efficacy of N1 and N2 immunization in naïve and LAC pre-infected mice (n=10-15 per condition). 3f, IsdB binding (ELISA) by biotinylated mAbs (0.5 μg/ml) in the absence or presence of IsdB-specific Ab (LAC-infected). 3g, Efficacy of adoptively transferred anti-IsdB mAbs (FIG. 2f, 0.5 mg/kg i.p.) against SA challenge in naïve and SA pre-infected mice (n=7-10 per condition). Each data point represents one mouse, bars denote median and dashed lines indicate the limit of detection. Error bars represent means±SD. Student's t test (3c, 3d and 3f) or one way ANOVA with Kruskal-Wallis test (3a, 3b and 3e to 3g).

FIGS. 4a-4f: Human anti-staphylococcal antibodies blunt protection conferred by IsdB and ClfA passive immunizations. 4a, Anti-IsdB IgG titer in adult human and naïve mouse sera (mice, n=4; humans, n=22). 4b, IsdB binding (ELISA) by biotinylated vaccine-generated IsdB-specific mouse Ab (N/IsdB, 1 μg) in the absence or presence of human IsdB-specific Ab (10 μg) (n=2). 4c, Anti-SA protection from co-transfer of IsdB-vaccinated mouse sera (50 μl i.v.) and sera from human donors (#1-3, 100 μl i.p.) (n=4-8 per condition). 4d, Anti-SA protection conferred by vaccine-generated (25 μg i.v.) IsdB-Ab in mice pre-transfused with control IgG or human IsdB-specific Ab (35 μg IV given 16 h prior). Each red square or circle represents mean SA burden from 3-5 mice (n=13 humans). 4e, ClfA binding (ELISA) by ClfA-immunized mouse serum (1:10⁶ dilution) in the presence of high ClfA-titer human sera (added 1 hr before the mouse serum). 4f, Anti-SA protection conferred by anti-ClfA human mAb (Tefibazumab, 300 μg i.p.) in mice pre-transfused with human sera with low or high titer anti-ClfA Ab (100 μl i.v. given 16 h prior) (n=5 per condition). Each data point represents serum from an individual mouse or human. Bars denote median and dashed lines indicate the limit of detection. Error bars represent means±SD. Student's t test (4e) or one way ANOVA with Kruskal-Wallis test (4a-4d and 4f).

FIGS. 5a-5c: IsdB immunization is not protective in mice previously infected with S. aureus. 5a, Serum IsdB-specific IgG levels in mice infected 1-3 times with SA (LAC) and bled at the indicated times. 5b, IsdB vaccination after 1-2 SA infections: Kidney SA burden from experiment FIG. 1c. (n=5 per condition). 5c, IsdB vaccination after 3 SA infections: Kidney SA burden from experiment FIG. 1d. (n=10 per condition). For (5a), data are plotted as mean. Each data point represents an individual mouse. One way ANOVA with Kruskal-Wallis test (5b and 5c).

FIGS. 6a-6c: Prior S. aureus infection induced by i.v. or subcutaneous inoculation blunts IsdB vaccine efficacy. 6a, Tissue bacterial burden in mice infected subcutaneously (twice 2 weeks apart) with SA (LAC), IsdB immunized two weeks later and then LAC challenged i.p. per FIG. 1a. (n=11-19 per condition). 6b, Tissue bacterial burden in mice infected once i.v. with SA, treated for 5 days with vancomycin and then immunized and LAC challenged i.p. per FIG. 1a. (n=9 per condition). 6c, Serum anti-IsdB IgG titer after 3 weekly SA nasal applications (n=9 per condition). Each data point represents an individual mouse; bars denote median and dashed lines indicate the limit of detection. One way ANOVA with Kruskal-Wallis test (6a to 6c).

FIGS. 7a-7e: Effect of varying S. aureus strain or mouse genetic background on IsdB vaccine interference. 7a-7b, Tissue bacterial burden in (C57BL/6) mice infected with SA Newman or SA113, then immunized and challenged with the same SA strain as per FIG. 1a. (n=4-5 per condition). 7c-7d, Tissue bacterial burden in BALB/c and CBA mice pre-infected with LAC, then immunized and LAC challenged as per FIG. 1a. (n=5 per condition). e, Tissue bacterial burden in male mice infected with LAC, then immunized and LAC challenged as per FIG. 1a. (n=9-10 per condition). Each data point represents an individual mouse; bars denote median and dashed lines indicate the limit of detection. One way ANOVA with Kruskal-Wallis test (7a to 7e).

FIGS. 8a-8c: Cellular source, persistence and specificity of IsdB vaccine interference. 8a, B cells adoptively transferred from SA (LAC) infected mice blunt IsdB vaccine efficacy in the recipient mice. Recipient mice were immunized with IsdB as per FIG. 1a, 24h after B cell transfer (n=10 per condition). 8b, Effect of extending the time interval between SA infection and IsdB vaccination to 21 days. Infection and immunization were otherwise performed as in FIG. 1a. (n=5 per condition). 8c, Specificity of vaccine suppression. Kidney SA burden from experiment FIG. 1f. (n=10 per condition). Each data point represents an individual mouse; bars denote median and dashed lines indicate the limit of detection. One way ANOVA with Kruskal-Wallis test (8a to 8c).

FIGS. 9a-9c: Anti-S. aureus function of adoptively transferred CD4+ T cells, B cells and total IgG purified from IsdB vaccinated S. aureus-naïve or pre-infected mice. 9a, B cell transfer: Kidney SA burden from experiment FIG. 1g. (n=10 per condition). 9b, Total IgG transfer: Kidney SA burden from experiment FIG. 1h. 9c, CD4+ T cell transfer from IsdB vaccinated SA-naïve mice is not protective (n=5 per condition). Each data point represents an individual mouse; bars denote median and dashed lines indicate the limit of detection. One way ANOVA with Kruskal-Wallis test (9a to 9c).

FIGS. 10a-10g: Characterization of IsdB-specific antibodies derived from S. aureus-infected mice and naïve or SA-infected mice immunized with IsdB. 10a, Titers of IsdB-specific Ig subclasses (IgM, IgG1, IgG2a, IgG2b, IgG2c and IgG3) in the sera of naïve or SA-infected mice at 1:1000 dilution. 10b-10d, Titers of IsdB-specific IgG1, IgG2b and IgG2c in the sera of mice treated as in FIG. 1a. 10e, Binding of purified anti-IsdB antibodies to WT LAC or isogenic spa mutant analyzed by flow cytometry. Grey: stain with FITC-conjugated anti-mouse IgG only. 10f, Ab-dependent complement deposition assay. 10g, Binding profile of sera to overlapping IsdB peptide array. Mouse sera: NT/IsdB: Naïve mice vaccinated with IsdB, LAC/IsdB: LAC-infected mice vaccinated with IsdB; LAC: LAC-infected mice given adjuvant alone (LAC/alum). NEAT1 extends from peptide #24 to #55, and NEAT2 from peptide #76 to #103. 10h, in vivo function of IsdB-specific mAbs from FIG. 2f: Kidney SA burden from experiment FIG. 2g. Data are plotted as mean±SD. Student's t test (10a to 10d and 10f) or one way ANOVA with Kruskal-Wallis test (10h).

FIGS. 11a-11b: IsdB-specific clonotypes and clonotype network of B cells derived from SA-infected mice and naïve or SA-infected mice immunized with IsdB. 11a, Sequences of the clonotypes from experiment FIG. 2h. 11b, Clonotype network: Each dot represents cells with identical CDR3 sequence. For each clonotype, the numeric clonotype ID is shown in the graph. The size of each dot refers to the number of cells with the same CDR3.

FIGS. 12a-12d: IsdB-specific antibody levels following vaccination of naïve and SA pre-infected mice. 12a, Serum IsdB-specific IgM level from experiment FIG. 2i. (n=10 per condition). 12b-12c, Serum IsdB-specific IgG and IgM levels in IsdB vaccinated WT and IsdB/HarA mutant-infected mice (n=10 per condition). 12d, Relative intensity of anti-IsdB IgG ASCs from experiment FIG. 2j. Each data point represents an individual mouse; bars denote median. One way ANOVA with Kruskal-Wallis test (12a to 12d).

FIGS. 13a-13e: Competition between non-protective and protective IsdB-specific antibodies determines outcome of staphylococcal infection. 13a, Anti-SA immunity conferred by protective sera (NT-IsdB) in the presence or absence of non-protective sera (LAC-IsdB): Kidney SA burden from experiment FIG. 3a. (n=8 per condition). 13b, Anti-SA immunity conferred by IsdB-specific protective Antibodies (NT-IsdB) in the presence or absence of non-protective specific antibodies (LAC-IsdB): Kidney SA burden from experiment FIG. 3b. (n=5-8 per condition). 13c, Serum IsdB-, NEAT1- and NEAT2-specific IgG levels following IsdB, N2 or N2 immunization of naïve mice respectively 13d, N2 vaccination confers anti-SA protection in naïve and SA pre-infected mice: Kidney SA burden from experiment FIG. 3f. (n=10-15 per condition). 13e, Anti-SA immunity conferred by anti-N2 mAbs in SA-pre-infected mice: Kidney SA burden from experiment FIG. 3h. (n=7-10 per condition). Data are plotted as mean±SD (c). Each data point represents an individual mouse; bars denote median. One way ANOVA with Kruskal-Wallis test (13a, 13b, 13d and 13e).

FIGS. 14a-14b: CDR3 sequences and IsdB epitope recognition by anti-IsdB monoclonal antibodies. 14a, Table of amino acid sequence of CDR3 of IsdB mAbs from FIG. 2f-g; 14b, Mapping of IsdB-specific mAb interaction with IsdB NEAT2 domain using peptide array. Peptide index of NEAT2: #76-#103.

FIGS. 15a-15c: Human serum anti-S. aureus antibodies blunt specific protection conferred by anti-IsdB immunization. 15a, In vivo human serum interference with protective anti-IsdB Ab function: Kidney SA burden from experiment FIG. 4c. (n=4-8 per condition). 15b, In vivo human IsdB-specific Ab interference with protective anti-IsdB Ab function: Kidney SA burden from experiment FIG. 4d. (human serum, n=13). 15c, In vivo human IsdB-specific Ab interference with protective anti-IsdB Ab function: Inhibition data by individual human antibody samples. Each data point represents an individual mouse; bars denote median and dashed lines indicate the limit of detection (15a and 15b). One way ANOVA with Kruskal-Wallis test (15a and 15b).

FIGS. 16a-16c: Human serum anti-S. aureus antibodies blunt specific protection conferred by anti-ClfA passive immunization. 16a, ClfA binding by ClfA-immunized mouse serum in the presence of human sera depleted of anti-ClfA antibodies (added 1 hr prior to mouse serum addition), in an ELISA plate assay. 16b, ClfA-specific IgG level in high (aCifA-Hi) and low titer (aClfA-lo) human sera. 16c, In vivo human serum interference with anti-ClfA mAb function: Kidney SA burden from experiment FIG. 4f. Data are plotted as mean±SD. One way ANOVA with Kruskal-Wallis test (16c).

### DETAILED DESCRIPTION OF EMBODIMENTS

### Abbreviations and Definitions

To facilitate understanding of the invention, a number of terms and abbreviations as used herein are defined below as follows:

Pharmaceutically acceptable carrier: As used herein, the term “Pharmaceutically acceptable carrier” refers to a diluent, adjuvant, excipient, or vehicle with which a compound is administered. Such carriers can be sterile liquids, such as water and oils, including those of petroleum, animal, vegetable, or synthetic origin, such as peanut oil, soybean oil, mineral oil, sesame oil and the like, polyethylene glycols, glycerine, propylene glycol, or other synthetic solvents. Water is a preferred carrier when a compound is administered intravenously. Saline solutions and aqueous dextrose and glycerol solutions can also be employed as liquid carriers, particularly for injectable solutions. Suitable excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol, and the like. A compound, if desired, can also combine minor amount of wetting or emulsifying agents, or pH buffering agents such as acetates, citrates, or phosphates. Antibacterial agents such as a benzyl alcohol or methyl parabens; antioxidants such as ascorbic acid or sodium bisulfite; chelating agents such as ethylenediaminetetraacetic acid; and agents for the adjustment of tonicity such as sodium chloride or dextrose may also be a carrier. Methods for producing compounds in combination with carriers are known to those of skill in the art.

Therapeutically effective amount: As used herein, the term “Therapeutically effective amount” refers to those amounts that, when administered to a particular subject in view of the nature and severity of that subject's disease or condition, will have a desired therapeutic effect, e.g. an amount that will cure, prevent, inhibit, or at least partially arrest or partially prevent a target disease or condition.

Polynucleotide Variant: A “polynucleotide variant” refers to any degenerate nucleotide sequence. Changes in the nucleotide sequence of the variant may or may not alter the amino acid sequence of a polypeptide encoded by the reference polynucleotide. Nucleotide changes may result in amino acid substitutions, additions, deletions, fusions and truncations in the polypeptide encoded by the reference sequence. For example, a variant polynucleotide consisting of 80, 81, 82, 83, 84, 85, 86, 87, 88, 89, 90, 91, 92, 93, 94, 95, 96, 97, 98, and 99% to the polynucleotide consisting of NEAT2.

Polynucleotide Fragment: A “polynucleotide fragment” of a NEAT2 polynucleotide is a portion of a NEAT2 polynucleotide that is less than full-length and comprises at least a minimum length capable of hybridizing specifically with a native NEAT2 polynucleotide under stringent hybridization conditions. The length of such a fragment is preferably at least 15 nucleotides, more preferably at least 20 nucleotides, and most preferably at least 30 nucleotides of a native NEAT2 polynucleotide sequence.

Polypeptide Variant: A “polypeptide variant” refers to a polypeptide of differs in amino acid sequence from the NEAT2 polypeptide. Generally, differences are limited so that the sequences of the reference polypeptide and the variant are closely similar overall and, in many regions, identical. A variant polypeptide may differ in amino acid sequence by one or more substitutions, additions, deletions in any combination. A substituted or inserted amino acid residue may or may not be one encoded by the polynucleotide code.

Polypeptide Fragment: A “polypeptide fragment” refers to any polypeptide of a portion of a NEAT2 polypeptide that is less than full-length (e.g., a polypeptide consisting of 5, 10, 15, 20, 30, 40, 50, 75, 100 or more amino acids of a native NEAT2 protein of SEQ ID NO: 1), and preferably retains at least one functional activity of a native NEAT2 protein.

Staphylococcus aureus Vaccine

The failure of all S. aureus (SA) vaccine trials to date prompted an exploration of the fundamental difference between humans and laboratory animals—their natural exposure to SA. Recapitulating the failed major IsdB vaccine trial, it was shown that mice previously infected with SA do not mount a protective vaccine antibody response, unlike naïve animals. SA infection alone induces non-protective anti-IsdB antibodies with enhanced α2,3 sialylation and specificity largely against a non-neutralizing IsdB domain. With prior SA infection, IsdB vaccination recalls the non-protective humoral response, which further competes and reduces efficacy of protective vaccine-generated specific antibodies. It has been shown that human serum antibodies against IsdB and another failed vaccine target, ClfA, blocked anti-staphylococcal immunity conferred by passive immunizations.

All S. aureus vaccines taken to clinical trials (over 15 of them) have failed even though the vaccines work in animal models. Rodent models are naive to human S. aureus whereas humans are exposed in the first few months of life to S. aureus. As provided herein, rodents exposed to S. aureus, S. aureus vaccines (normally protective vaccines, such as the IsdB vaccine), are ineffective because of immune recall of non-protective immunity against S. aureus.

Therefore, the present invention is directed to a new strategy of making a S. aureus IsdB vaccine: by targeting the NEAT2 IsdB subdomain (SEQ ID NO: 1) rather than the whole IsdB protein. The following Tables 1 and 2 respectively provide the amino acid sequence of the NEAT2 protein and NEAT2 polynucleotide sequence useful in immunizations:

TABLE 1
NEAT2 Amino Acid Sequence (SEQ ID NO: 1)

| | |
|---|---|
| TEEDYKAEKL LAPYKKAKTL ERQVYELNKI QDKLPEKLKA EYKKKLEDTK KALDEQVKSA | 60 |
| ITEFQNVQPT NEKMTDLQDT KYVVYESVEN NESMMDTFVK HPIKIGMLNG KKYMVMETTN | 120 |
| DDYWKDFMVE GQRVRTISKD AKNNTRTIIF PYVEGKTLYD AIVKVHVKTI DYDGQYHVRI | 180 |
| VDKEAFTKAN TDKSNKKEQQ DNSAKKEATP ATPSKPTPSP VEKESQKQDS QKDDNKQLPS | 240 |
| VEKENDASSE SGKDKTPATK PTKGEVESSS TTPTKVVSTT QNVAKPTTAS SKTTKDVVQT | 300 |
| SAGESEAKDS APLQKANIKN TNDGHTQSQN NKNTQENKAK SLPQTGEESN KDMTLPLMAL | 360 |
| LALSSIVAFV LPRKRKN | 377 |

TABLE 2
NEAT2 Nucleotide Sequence (SEQ ID NO: 2)

| | |
|---|---|
| actgaagaag attataaagc tgaaaaatta ttagcgccat ataaaaaagc gaaaacacta | 60 |
| gaaagacaag tttatgaatt aaataaaatt caagataaac ttcctgaaaa attaaaggct | 120 |
| gagtacaaga agaaattaga ggatacaaag aaagctttag atgagcaagt gaaatcagct | 180 |
| attactgaat tccaaaatgt acaaccaaca aatgaaaaaa tgactgattt acaagataca | 240 |
| aaatatgttg tttatgaaag tgttgagaat aacgaatcta tgatggatac ttttgttaaa | 300 |
| caccctatta aaacaggtat gcttaacggc aaaaaatata tggtcatgga aactactaat | 360 |
| gacgattact ggaaagattt catggttgaa ggtcaacgtg ttagaactat aagcaaagat | 420 |
| gctaaaaata atactagaac aattattttc ccatatgttg aaggtaaaac tctatatgat | 480 |
| gctatcgtta aagttcacgt aaaaacgatt gattatgatg gacaatacca tgtcagaatc | 540 |
| gttgataaag aagcatttac aaaagccaat accgataaat ctaacaaaaa agaacaacaa | 600 |
| gataactcag ctaagaagga agctactcca gctacgccta gcaaaccaac accatcacct | 660 |
| gttgaaaaag aatcacaaaa acaagacagc caaaaagatg acaataaaca attaccaagt | 720 |
| gttgaaaaag aaaatgacgc atctagtgag tcaggtaaag acaaaacgcc tgctacaaaa | 780 |
| ccaactaaag gtgaagtaga atcaagtagt acaactccaa ctaaggtagt atctacgact | 840 |
| caaaatgttg caaaaccaac aactgcttca tcaaaaacaa caaaagatgt tgttcaaact | 900 |
| tcagcaggtt ctagcgaagc aaaagatagt gctccattac aaaaagcaaa cattaaaaac | 960 |
| acaaatgatg gacacactca aagccaaaac aataaaaata cacaagaaaa taaagcaaaa | 1020 |
| tcattaccac aaactggtga agaatcaaat aaagatatga cattaccatt aatggcatta | 1080 |
| ttagctttaa gtagcatcgt tgcattcgta ttacctagaa aacgtaaaaa ctaa | 1134 |

Variants and fragments of these sequences are also useful and those of skill in the art are able to determine which of the variants and fragments have at least one activity of SEQ ID NOs: 1 and 2 by referencing the Examples provided herein.

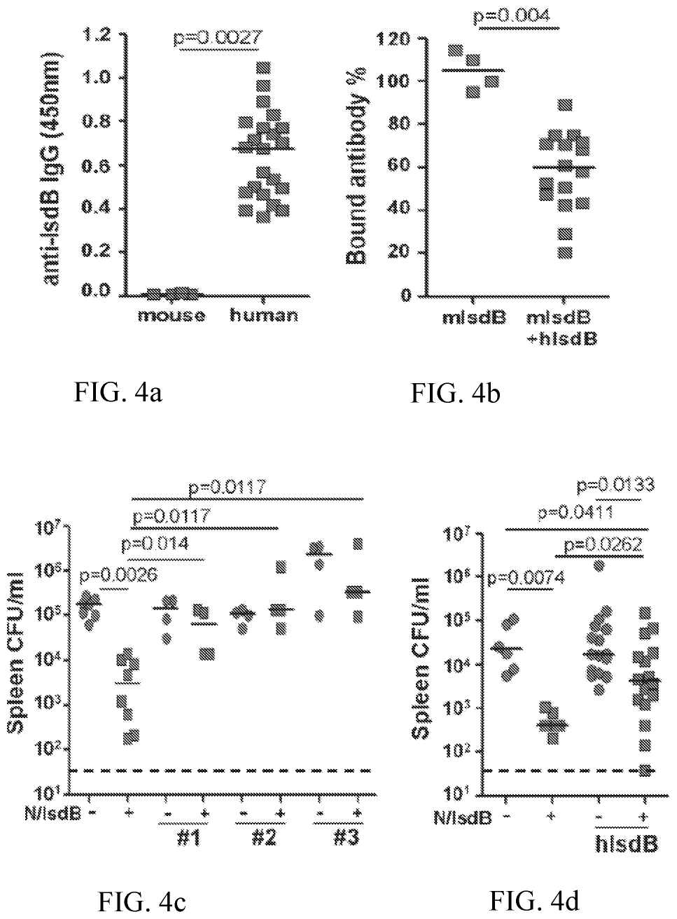

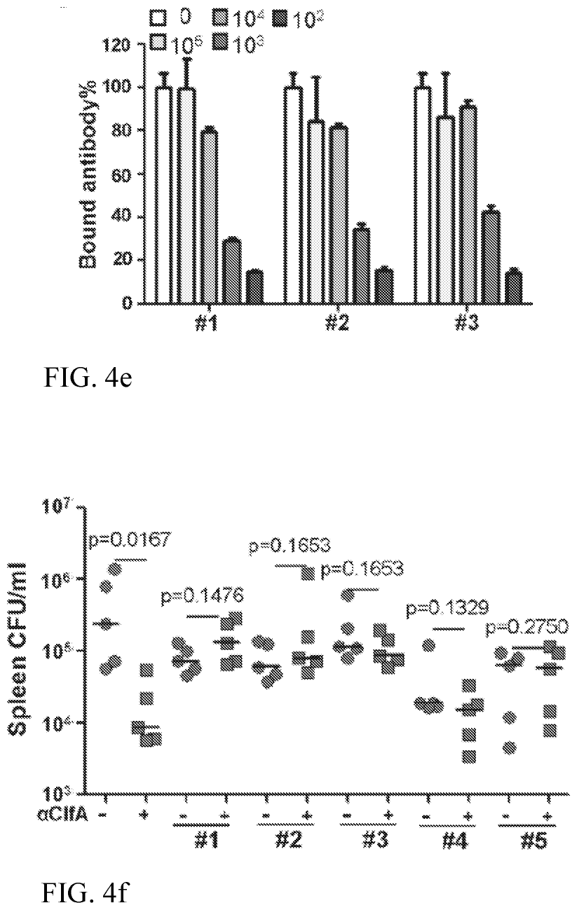

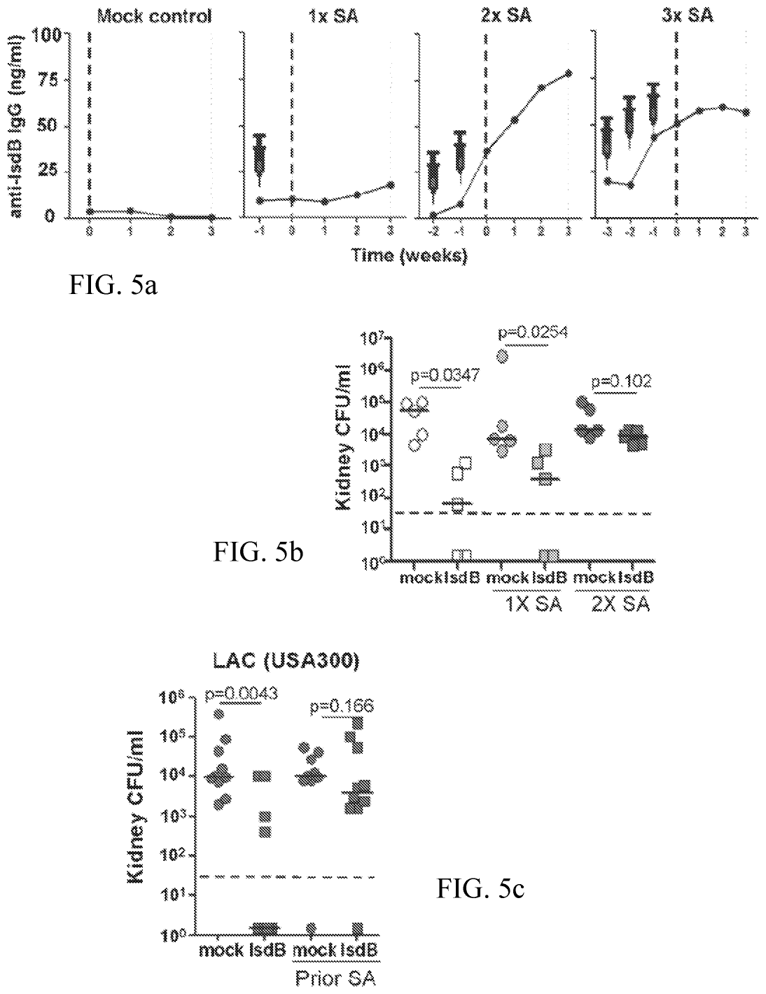

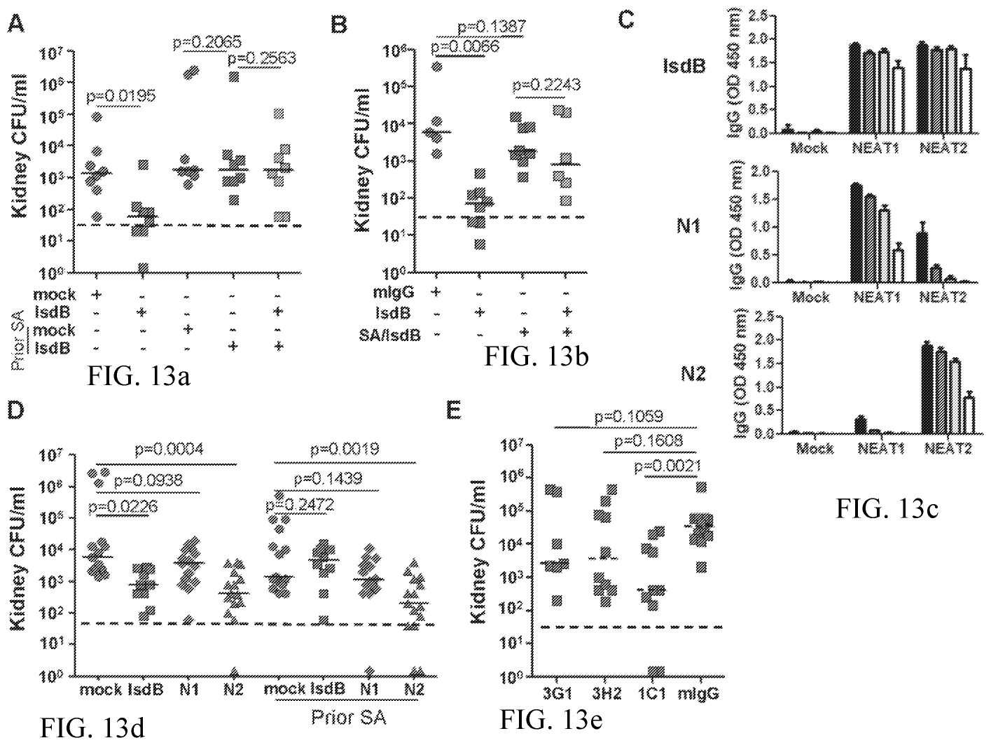

Provided herein, it is shown that if a subject is immunized with a subdomain of IsdB, NEAT2, the IsdB vaccine is effective again, because of avoidance of antibody interference. See, e.g., FIGS. 4E and 13 C and D.

Prior S. aureus infection induces non-protective human or mouse IsdB antibody response, which is described more fully herein. IsdB vaccination of S. aureus exposed mice recalls the non-protective antibody response, which weakens the efficacy of a vaccine response.

Furthermore, the non-protective antibodies against IsdB compete against the protective IsdB response for Fab and F, binding, thereby blocking vaccine protection. By vaccinating against the protective NEAT2 domain of IsdB, recall of much of the non-protective antibodies against IsdB is avoided, thereby avoiding interference.

Protection Against S. aureus

In a preferred embodiment of the invention, an immunogenic vaccine composition provides an effective immune response against SA, and more preferably, more than one strain of staphylococci.

An effective immune response is defined as an immune response that gives significant protection in a mouse challenge model as described in the examples. Significant protection in a mouse challenge model is defined as an increase in the LD₅₀ in comparison with carrier inoculated mice of at least 10%, 20%, 50%, 100% or 200%. The presence of opsonizing antibodies is known to correlate with protection, therefore significant protection is indicated by a decrease in the bacterial count of at least 10%, 20%, 50%, 70% or 90% in an opsonophagocytosis assay, described in more detail in the Examples.

### Vaccines

In an embodiment, the immunogenic vaccine composition of the invention is mixed with a pharmaceutically acceptable carrier, and more preferably with an adjuvant to form a vaccine.

The vaccines of the present invention are preferably adjuvanted. Suitable adjuvants include an aluminum salt such as aluminum hydroxide gel (alum) or aluminum phosphate, but may also be a salt of calcium, magnesium, iron or zinc, or may be an insoluble suspension of acylated tyrosine, or acylated sugars, cationically or anionically derivatized polysaccharides, or polyphosphazenes.

Optionally, the adjuvant may be selected to be a preferential inducer of either a TH1 or a TH2 type of response. High levels of Th1-type cytokines tend to favor the induction of cell mediated immune responses to a given antigen, while high levels of Th2-type cytokines tend to favor the induction of humoral immune responses to the antigen.

The vaccine composition comprises a NEAT2 polypeptide comprising an amino acid sequence corresponding to SEQ ID NO: 1. The vaccine composition can further comprise a polypeptide including, but not limited to, STAPHVAX manufactured by NABI, ALTASTAPH manufactured by NABI, PENTASTAPH manufactured by NABI/GSK, AUROGRAB manufactured by Novartis, VERONATE manufactured by Inhibitex, Tefibazumab manufactured by Inhibitex, Pagibaximab manufactured by Biosynexus, V710 manufactured by Merck, SAR279356 manufactured by Sanofi, NVD3 manufactured by Novadigm, STEBVAX manufactured by IBT, SA3Ag manufactured by Pfizer, PF-06290510 manufactured by Pfizer, and MEDI4893 manufactured by Medimmune. Each of the above compositions is more fully described at https://clinicaltrials.gov.

Such combinations may be administered simultaneously. More preferably, however, the NEAT2 vaccine composition will be administered prior to the polypeptides above. If the polypeptides above are administered first, then the NEAT2 vaccine composition can be administered second such that the second administration of the polypeptides above will show enhanced effect.

The vaccine composition may further comprise one or more adjuvants. Suitable adjuvants are known in the art and include, without limitation, flagellin, Freund's complete or incomplete adjuvant, aluminum hydroxide, lysolecithin, pluronic polyols, polyanions, peptides, oil emulsion, dinitrophenol, iseomatrix, and liposome polycation DNA particles.

The vaccine composition as described herein may be prepared by formulating the recombinantly produced NEAT2 variants or fragments with a pharmaceutically acceptable carrier and optionally a pharmaceutically acceptable excipient.

Another aspect of the present disclosure relates to a method of immunizing a subject against an SA infection. This method involves administering the vaccine composition comprising the NEAT2 polypeptide, or variants or fragments, in an amount effective to immunize against SA infection in the subject. A suitable subject for treatment in accordance with this aspect of the present invention is a subject at risk of developing a SA infection.

In accordance with this aspect, a therapeutically effective amount of the vaccine composition for administration to a subject to immunize against S. aureus infection is the amount necessary to generate a humoral (i.e., antibody mediated) immune response. The generated humoral response is sufficient to prevent or at least reduce the extent of S. aureus infection that would otherwise develop in the absence of such response. Preferably, administration of a therapeutically effective amount of the vaccine composition described herein induces a neutralizing immune response against S. aureus in the subject. To effectuate an effective immune response in a subject, the composition may further contain one or more additional S. aureus antigens or an adjuvant as described above. In an alternative embodiment, the adjuvant is administered separately from the composition to the subject, either before, after, or concurrent with administration of the composition of the present invention.

For purposes of this aspect the disclosure, the target “subject” encompasses any animal, preferably a mammal, more preferably a human. In the context of administering a vaccine composition for purposes of preventing a S. aureus infection in a subject, the target subject encompasses any subject that is at risk of being infected by S. aureus. Particularly susceptible subjects include infants and juveniles, as well as immunocompromised juvenile, adults, and elderly adults. However, any infant, juvenile, adult, or elderly adult or immunocompromised individual at risk for S. aureus infection can be treated in accordance with the methods and vaccine composition described herein. Particularly suitable subjects include those at risk of infection with methicillin-resistant S. aureus (MRSA) or methicillin sensitive S. aureus (MSSA).

Therapeutically effective amounts of the vaccine composition comprising NEAT2, or variants or fragments thereof, for immunization will depend on whether an adjuvant is co-administered, with higher dosages being required in the absence of adjuvant. The amount of NEAT2 polypeptide, including variants and fragments, for administration sometimes varies from 1 μg-500 μg per patient and more usually from 5-500 μg per injection for human administration. Occasionally, a higher dose of 1-2 mg per injection is used. Typically about 10, 20, 50 or 100 μg is used for each human injection. The timing of injections can vary significantly from once a day, to once a year, to once a decade. Generally an effective dosage can be monitored by obtaining a fluid sample from the subject, generally a blood serum sample, and determining the titer of antibody developed against NEAT2, using methods well known in the art and readily adaptable to the specific antigen to be measured. Ideally, a sample is taken prior to initial dosing and subsequent samples are taken and titered after each immunization. Generally, a dose or dosing schedule which provides a detectable titer at least four times greater than control or “background” levels at a serum dilution of 1:100 is desirable, where background is defined relative to a control serum or relative to a plate background in ELISA assays.

Adjuvants to enhance effectiveness of the composition include, but are not limited to: (1) oil-in-water emulsion formulations (with or without other specific immunostimulating agents such as muramyl peptides (see below) or bacterial cell wall components), such as for example (a) MF59™ (see Chapter 10 in Vaccine design: the subunit and adjuvant approach, eds. Powell & Newman, Plenum Press 1995), containing 5% Squalene, 0.5% Tween 80, and 0.5% Span 85 (optionally containing various amounts of MTP-PE (see below), although not required) formulated into submicron particles using a microfluidizer such as Model 110Y microfluidizer (Microfluidics, Newton, Mass.), (b) SAF, containing 10% Squalene, 0.4% Tween 80, 5% pluronic-blocked polymer L121, and thr-MDP (see below) either microfluidized into a submicron emulsion or vortexed to generate a larger particle size emulsion, and (c) Ribi™ adjuvant system (RAS), (Ribi Immunochem, Hamilton, Mont.) containing 2% Squalene, 0.2% Tween 80, and one or more bacterial cell wall components from the group consisting of monophosphorylipid A (MPL), trehalose dimycolate (TDM), and cell wall skeleton (CWS), preferably MPL+CWS (Detox™); (2) saponin adjuvants, such as Stimulon™ (Cambridge Bioscience, Worcester, Mass.) may be used or particles generated therefrom such as ISCOMs (immunostimulating complexes); (3) Complete Freund's Adjuvant (CFA) and Incomplete Freund's Adjuvant (IFA); (4) cytokines, such as interleukins (e.g., IL-1, IL-2, IL-4, IL-5, IL-6, IL-7, IL-12, etc.), interferons (e.g., gamma interferon), macrophage colony stimulating factor (M-CSF), tumor necrosis factor (TNF), etc.; (5) detoxified mutants of a bacterial ADP-ribosylating toxin such as a cholera toxin (CT), a pertussis toxin (PT), or an E. coli heat-labile toxin (LT), particularly Lt-K63, LT-R72, CT-S109, PT-K9/G129; and (6) other substances that act as immunostimulating agents to enhance the effectiveness of the composition.

As mentioned above, muramyl peptides include, but are not limited to, N-acetyl-muramyl-L-threonyl-Disoglutamine (thr-MDP), N-acetyl-normuramyl-L-alanyl-D-isoglutamine (nor-MDP), N-acetylmuramyl-L-alanyl-D-isoglutaminyl-L-alanine-2-(1′-2′-dipalmitoyl-s-n-glycero-3-hydroxyphosphoryloxy)-ethylamine (MTP-PE), etc.

The vaccine compositions comprising immunogenic compositions (e.g., which may include the antigen, pharmaceutically acceptable carrier, and adjuvant) typically will contain diluents, such as water, saline, glycerol, ethanol, etc. Additionally, auxiliary substances, such as wetting or emulsifying agents, pH buffering substances, and the like, may be present in such vehicles. Alternatively, vaccine compositions comprising immunogenic compositions may comprise an antigen, polypeptide, protein, protein fragment or nucleic acid in a pharmaceutically acceptable carrier.

More specifically, vaccines comprising immunogenic compositions comprise an immune response activating amount of the immunogenic polypeptides, as well as any other of the above-mentioned components, as needed. It is expected that the amount will fall in a relatively broad range that can be determined through routine trials.

Typically, the vaccine compositions or immunogenic compositions are prepared as injectables, either as liquid solutions or suspensions. Solid forms suitable for solution in, or suspension in, liquid vehicles prior to injection may also be prepared. The preparation also may be emulsified or encapsulated in liposomes for enhanced adjuvant effect, as discussed above under pharmaceutically acceptable carriers.

The immunogenic compositions are conventionally administered parenterally, e.g., by injection, either subcutaneously, intramuscularly, or transdermally/transcutaneously. Additional formulations suitable for other modes of administration include oral and pulmonary formulations, suppositories, and transdermal applications. Dosage treatment may be a single dose schedule or a multiple dose schedule. The vaccine may be administered in conjunction with other immunoregulatory agents.

Although the vaccine of the present invention may be administered as a single dose, components thereof may also be co-administered together at the same time or at different times (for instance pneumococcal polysaccharides could be administered separately, at the same time or 1-2 weeks after the administration of any bacterial protein component of the vaccine for optimal coordination of the immune responses with respect to each other). For co-administration, an optional Th1 adjuvant may be present in any or all of the different administrations, however it is preferred if it is present in combination with the bacterial protein component of the vaccine. In addition to a single route of administration, two different routes of administration may be used. For example, polysaccharides may be administered IM (or ID) and bacterial proteins may be administered IN (or ID). In addition, the vaccines of the invention may be administered IM for priming doses and IN for booster doses.

Vaccine preparation is generally described in Vaccine Design (“The subunit and adjuvant approach” (eds Powell M. F. & Newman M. J.) (1995) Plenum Press New York). Encapsulation within liposomes is described by Fullerton, U.S. Pat. No. 4,235,877.

The vaccines of the present invention may be stored in solution or lyophilized. Preferably the solution is lyophilized in the presence of a sugar such as sucrose, trehalose or lactose. It is still further preferable that they are lyophilized and extemporaneously reconstituted prior to use.

### Polynucleotide Vaccines

mRNA vaccines have become well known in the art. Hence, the disclosures of U.S. Pat. Nos. 11,622,972, 11,524,023, 11,485,972, 10,898,574, 10,703,789, 10,702,600, 10,577,403, 10,442,756, 10,266,485, 10,064,959, and 9,868,692, each assigned to Moderna, Inc., are incorporated herein by reference to the fullest extent possible. These disclosures provide those of skill in the art detailed descriptions of incorporating the polynucleotide sequence of SEQ ID NO: 2, or variant or fragment thereof, into a system form immunizing subjects against S. aureus infection.

Similarly, the disclosures of U.S. Pat. Nos. 10,576,146, 10,485,884, and 9,950,065 as well as US Pat. App. Nos. US2020/0155671, US2020/0197508, US2019/0153428, US2019/0321458, US2018/0263907, US2017/0273907, US2014/0030808, WO2016/156398, WO2015/043613, and WO2013/087083, each assigned to BioNTech SE, are incorporated herein by reference to the fullest extent possible. These disclosures provide those of skill in the art detailed descriptions of incorporating the polynucleotide sequence of SEQ ID NO: 2, or variant or fragment thereof, into a system form immunizing subjects against S. aureus infection.

One of skill in the art will understand how to incorporate NEAT2 polynucleotide, including variants and fragments thereof, into the Moderna or BioNTech systems described in the patents and patent applications above. Similar to introducing a COVID-19 spike protein polynucleotide into those systems, one of skill in the art will readily understand how to introduce NEAT2 into those systems.

In a further aspect, the present invention relates to the use of the polynucleotide sequence of SEQ ID NO: 2, or variant or fragment thereof, in the treatment, prevention or diagnosis of staphylococcal infection. Such polynucleotides include isolated polynucleotides comprising a nucleotide sequence encoding a polypeptide which has at least 70% identity, preferably at least 80% identity, more preferably at least 90% identity, yet more preferably at least 95% identity, to the amino acid sequence of SEQ ID NO: 2, over the entire length of the sequence. In this regard, polypeptides which have at least 95% identity are highly preferred, while those with at least 96-97% identity are more highly preferred, even while those with at least 98-99% identity are even more highly preferred, and those with at least 99% identity are most highly preferred.

Further polynucleotides that find utility in the present invention include isolated polynucleotides comprising a nucleotide sequence that has at least 70% identity, preferably at least 80% identity, more preferably at least 90% identity, yet more preferably at least 95% identity, to a nucleotide sequence encoding a NEAT2 polypeptide, or variant or fragment thereof, of the invention over the entire coding region. In this regard, polypeptides which have at least 95% identity are highly preferred, while those with at least 96-97% identity are more highly preferred, even while those with at least 98-99% identity are even more highly preferred, and those with at least 99% identity are most highly preferred. Such polynucleotide can be inserted in a suitable plasmid or recombinant microorganism vector and used for immunization.

The present invention also provides a nucleic acid encoding the aforementioned proteins of the present invention and their use in medicine. In a preferred embodiment isolated polynucleotides according to the invention may be single-stranded (coding or antisense) or double-stranded, and may be DNA (genomic, cDNA or synthetic) or RNA molecules. Additional coding or non-coding sequences may, but need not, be present within a polynucleotide of the present invention. In other related embodiments, the present invention provides polynucleotide variants having substantial identity to the sequences disclosed herein in SEQ ID NO: 2; those comprising at least 70% sequence identity, preferably at least 75%, 80%, 85%, 90%, 95%, 96%, 97%, 98%, or 99% or higher, sequence identity compared to a polynucleotide sequence of this invention using the methods described herein, (e.g., BLAST analysis using standard parameters). In a related embodiment, the isolated polynucleotide of the invention will comprise a nucleotide sequence encoding a polypeptide that has at least 90%, preferably 95% and above, identity to the amino acid sequence of SEQ ID NO: 1, over the entire length of a sequence of SEQ ID NO: 1; or a nucleotide sequence complementary to said isolated polynucleotide.

The invention also contemplates the use of polynucleotides which are complementary to all the above described polynucleotides.

The invention also provides for the use of a fragment of a polynucleotide of the invention which when administered to a subject has the same immunogenic properties as a polynucleotide of SEQ ID NO: 2.

The invention also provides for the use of a polynucleotide encoding an immunological fragment of a protein of SEQ ID NO: 1 as defined above. Also contemplated are the use of such fragments that have a level of immunogenic activity of at least about 50%, preferably at least about 70% and more preferably at least about 90% of the level of immunogenic activity of a polypeptide sequence encoded by a polynucleotide sequence set forth in SEQ ID NO: 2.

Polypeptide fragments for use according to the invention preferably comprise at least about 5, 10, 15, 20, 25, 50, or 100 contiguous amino acids, or more, including all intermediate lengths, of a polypeptide composition set forth herein, such as those set forth above.

Polynucleotides for use in the invention may be obtained, using standard cloning and screening techniques, from a cDNA library derived from mRNA in cells of human preneoplastic or tumor tissue (lung for example), (for example Sambrook et al., Molecular Cloning: A Laboratory Manual, 2nd Ed., Cold Spring Harbor Laboratory Press, Cold Spring Harbor, N.Y. (1989)). Polynucleotides of the invention can also be obtained from natural sources such as genomic DNA libraries or can be synthesized using well-known and commercially available techniques.

The polynucleotides of the disclosure may be produced by chemical synthesis such as solid phase polynucleotide synthesis on an automated polynucleotide synthesizer and assembled into complete single or double stranded molecules. Alternatively, the polynucleotides of the disclosure may be produced by other techniques such a PCR followed by routine cloning. Techniques for producing or obtaining polynucleotides of a given known sequence are well known in the art.

The polynucleotides described herein may comprise at least one non-coding sequence, such as a promoter or enhancer sequence, intron, polyadenylation signal, a cis sequence facilitating RepA binding, and the like. The polynucleotide sequences may also comprise additional sequences encoding additional amino acids that encode for example a marker or a tag sequence such as a histidine tag or an HA tag to facilitate purification or detection of the protein, a signal sequence, a fusion protein partner such as RepA, Fc or bacteriophage coat protein such as pIX or pIII.

Another embodiment of the disclosure is a vector comprising at least one polynucleotides as described herein. Such vectors may be plasmid vectors, viral vectors, vectors for baculovirus expression, transposon based vectors or any other vector suitable for introduction of the polynucleotides of the invention into a given organism or genetic background by any means. Such vectors may be expression vectors comprising nucleic acid sequence elements that can control, regulate, cause or permit expression of a polypeptide encoded by such a vector. Such elements may comprise transcriptional enhancer binding sites, RNA polymerase initiation sites, ribosome binding sites, and other sites that facilitate the expression of encoded polypeptides in a given expression system. Such expression systems may be cell-based, or cell-free systems well known in the art.

Vectors comprising such DNA, hosts transformed thereby and the truncated or hybrid proteins themselves, expressed as described herein all form part of the invention.

The expression system may also be a recombinant live microorganism, such as a virus or bacterium. The gene of interest can be inserted into the genome of a live recombinant virus or bacterium. Inoculation and in vivo infection with this live vector will lead to in vivo expression of the antigen and induction of immune responses.

Therefore, in certain embodiments, polynucleotides encoding immunogenic polypeptides for use according to the present invention are introduced into suitable mammalian host cells for expression using any of a number of known viral-based systems. In one illustrative embodiment, retroviruses provide a convenient and effective platform for gene delivery systems. A selected nucleotide sequence encoding a polypeptide for use in the present invention can be inserted into a vector and packaged in retroviral particles using techniques known in the art. The recombinant virus can then be isolated and delivered to a subject.

In addition, a number of illustrative adenovirus-based systems have also been described.

Various adeno-associated virus (AAV) vector systems have also been developed for polynucleotide delivery. AAV vectors can be readily constructed using techniques well known in the art.

Additional illustrative information on these and other known viral-based delivery systems can be found, for example, in Fisher-Hoch et al., Proc. Natl. Acad. Sci. USA 86:317-321, 1989; Flexner et al., Ann. N.Y. Acad. Sci. 569:86-103, 1989; Flexner et al., Vaccine 8:17-21, 1990; U.S. Pat. Nos. 4,603,112, 4,769,330, and 5,017,487; WO 89/01973; U.S. Pat. No. 4,777,127; GB 2,200,651; EP 0,345,242; WO 91/02805; Berkner, BioTechniques 6:616-627, 1988; Rosenfeld et al., Science 252:431-434, 1991; Kolls et al., Proc. Natl. Acad. Sci. USA 91:215-219, 1994; Kass-Eisler et al., Proc. Natl. Acad. Sci. USA 90:11498-11502, 1993; Guzman et al., Circulation 88:2838-2848, 1993; and Guzman et al., Cir. Res. 73:1202-1207, 1993.

The recombinant live microorganisms described above can be virulent, or attenuated in various ways in order to obtain live vaccines. Such live vaccines also form part of the invention.

In another embodiment of the invention, a polynucleotide is administered/delivered as “naked” DNA, for example as described in Ulmer et al., Science 259:1745-1749, 1993 and reviewed by Cohen, Science 259:1691-1692, 1993. The uptake of naked DNA may be increased by coating the DNA onto biodegradable beads, which are efficiently transported into the cells.

### Methods

The invention also encompasses method of making the immunogenic compositions and vaccines of the invention.

A preferred process of the invention is a method to make a vaccine comprising the steps of mixing antigens to make the immunogenic composition of the invention and adding a pharmaceutically acceptable excipient.

### Methods of Treatment

The invention also encompasses method of treatment or SA infection. The vaccine compositions of the present invention may be used to protect or treat a mammal susceptible to infection, by means of administering said vaccine via systemic or mucosal route. These administrations may include injection via the intramuscular, intraperitoneal, intradermal or subcutaneous routes; or via mucosal administration to the oral/alimentary, respiratory, genitourinary tracts.

The amount of antigen in each vaccine dose is selected as an amount which induces an immunoprotective response without significant, adverse side effects in typical vaccines. In various embodiments, such amount will vary depending upon which specific immunogen is employed and how it is presented. In one embodiment, the protein content of the vaccine will be in the range 1-100 μg, preferably 5-50 μg, most typically in the range 10-25 μg. Optionally, each dose can comprise 0.1-100 μg of polysaccharide where present. An optimal amount for a particular vaccine can be ascertained by standard studies involving observation of appropriate immune responses in subjects. Following an initial vaccination, subjects may receive one or several booster immunizations adequately spaced.

Although the vaccines of the present invention may be administered by any route, administration of the described vaccines into the skin (ID) forms one embodiment of the present invention. Human skin comprises an outer “horny” cuticle, called the stratum corneum, which overlays the epidermis. Underneath this epidermis is a layer called the dermis, which in turn overlays the subcutaneous tissue. Researchers have shown that injection of a vaccine into the skin, and in particular the dermis, stimulates an immune response, which may also be associated with a number of additional advantages. Intradermal vaccination with the vaccines described herein forms a preferred feature of the present invention.

The conventional technique of intradermal injection, the “mantoux procedure”, comprises steps of cleaning the skin, and then stretching with one hand, and with the bevel of a narrow gauge needle (26-31 gauge) facing upwards the needle is inserted at an angle of between 10-15°. Once the bevel of the needle is inserted, the barrel of the needle is lowered and further advanced while providing a slight pressure to elevate it under the skin. The liquid is then injected very slowly thereby forming a bump on the skin surface, followed by slow withdrawal of the needle.

Alternative methods of intradermal administration of the vaccine preparations may include conventional syringes and needles, or devices designed for ballistic delivery of solid vaccines, or transdermal patches; or applied to the surface of the skin (transdermal or transcutaneous delivery).

When the vaccines of the present invention are to be administered to the skin, or more specifically into the dermis, the vaccine is in a low liquid volume, particularly a volume of between about 0.05 ml and 0.2 ml.

The content of antigens in the skin or intradermal vaccines of the present invention may be similar to conventional doses as found in intramuscular vaccines (see above). However, it is a feature of skin or intradermal vaccines that the formulations may be “low dose”. Accordingly the protein antigens in “low dose” vaccines are preferably present in as little as 0.1 to 10 preferably 0.1 to 5 μg per dose; and the optional polysaccharide (preferably conjugated) antigens may be present in the range of 0.01-1 μg of polysaccharide per dose.

As used herein, the term “intradermal delivery” means delivery of the vaccine to the region of the dermis in the skin. However, the vaccine will not necessarily be located exclusively in the dermis. The dermis is the layer in the skin located between about 1.0 and about 2.0 mm from the surface in human skin, but there is a certain amount of variation between individuals and in different parts of the body. In general, it can be expected to reach the dermis by going 1.5 mm below the surface of the skin. The dermis is located between the stratum corneum and the epidermis at the surface and the subcutaneous layer below. Depending on the mode of delivery, the vaccine may ultimately be located solely or primarily within the dermis, or it may ultimately be distributed within the epidermis and the dermis.

A preferred embodiment of the invention is a method of preventing or treating SA infection or disease comprising the step of administering the vaccine composition or vaccine of the invention to a subject in need thereof.

A further preferred embodiment of the invention is a use of the vaccine composition of the invention in the manufacture of a vaccine for treatment or prevention of SA infection.

### DISCUSSION

Prior S. aureus Infection Abrogates Protection Conferred by IsdB Vaccine.

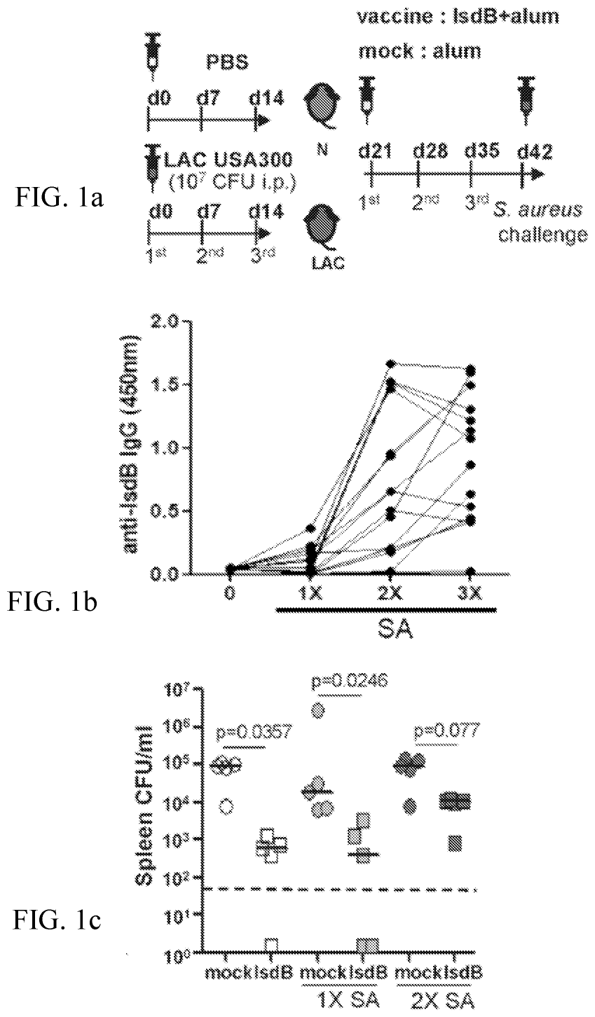

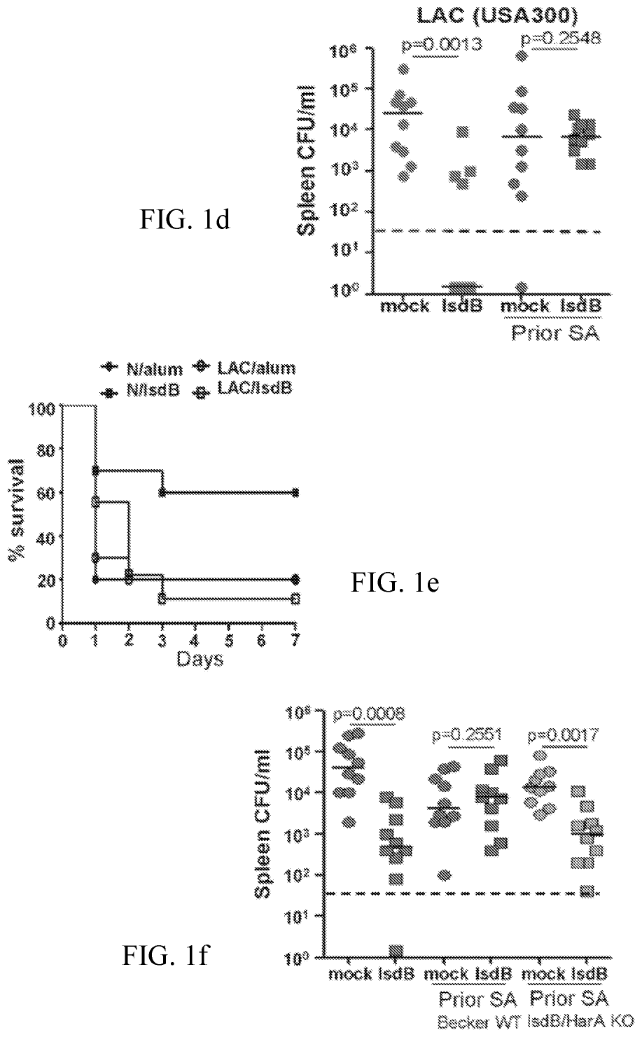

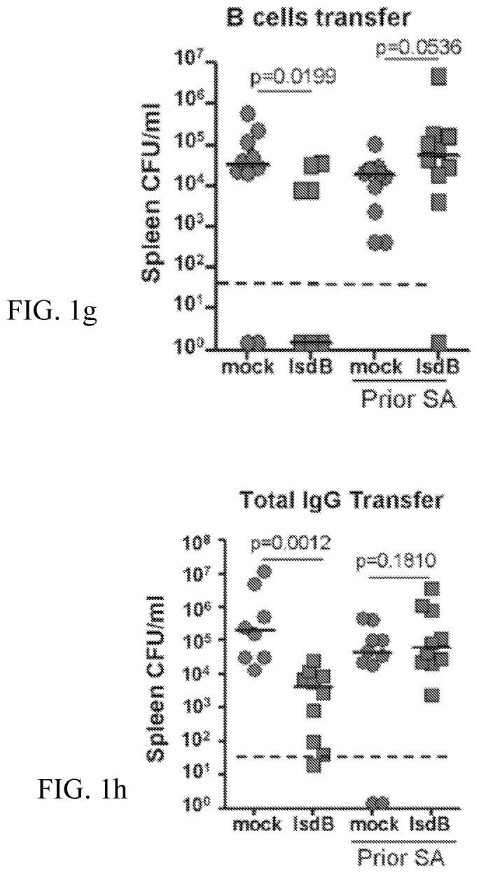

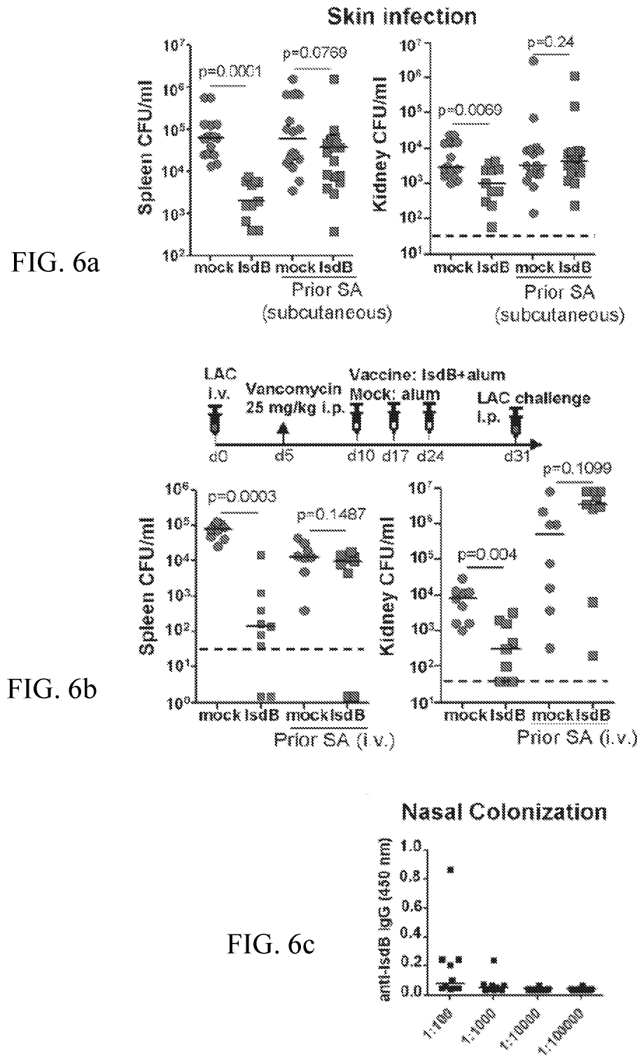

To model prior host exposure to SA, we intraperitoneally (i.p,) injected C57BL/6 mice with SA 1-3 times at weekly intervals with an aim to achieve serum IsdB antibody titers that approximate human serum levels (FIG. 6a). We then immunized the mice with recombinant IsdB plus alum and evaluated for bacterial burden or mouse mortality (FIG. 1a).

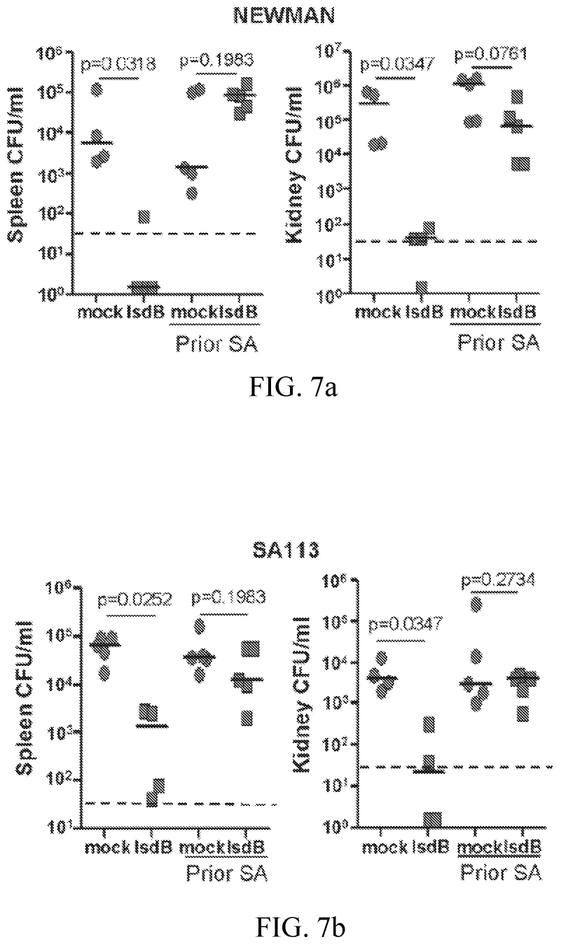

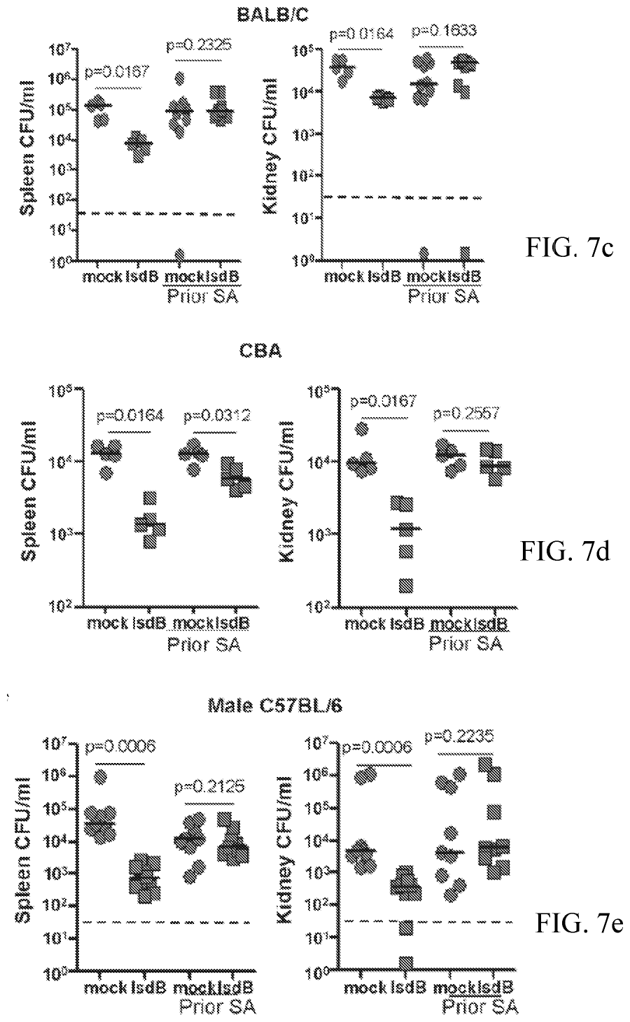

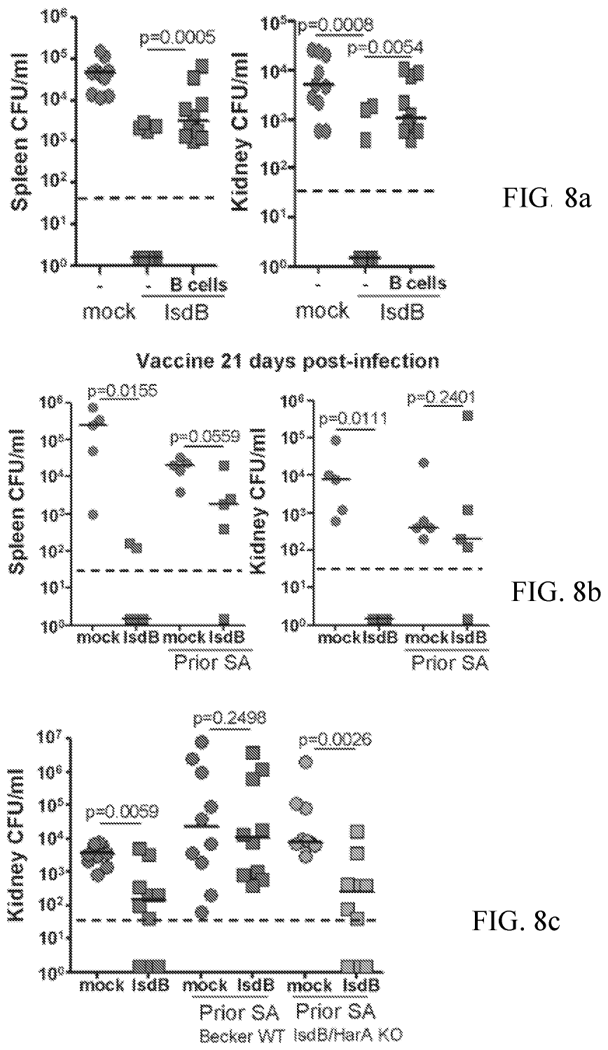

Corroborating earlier studies¹³, IsdB vaccine was highly efficacious against SA challenge in naïve mice (FIG. 1c). However, prior SA infections attenuated anti-SA immunity conferred by IsdB vaccine in direct correlation with pre-existing IsdB Ab titers (FIGS. 1b-e, FIGS. 6b-c). Vaccine efficacy was also blunted by prior SA infections delivered subcutaneously or i.v. (FIGS. 7a-b). Unlike human staphylococcal colonization, murine intranasal colonization with SA over 3 weeks failed to induce significant anti IsdB-specific Ab response (FIG. 7c). Hence, the effect of intranasal infection on IsdB vaccine was not studied. IsdB vaccine interference was observed in mice inoculated with SA strains LAC, SA113 or Newman when used for both prior infection and final challenge (FIG. 1d, FIGS. 8a-b). Interference was dramatic in all mouse genetic backgrounds tested (C57BL/6, CBA, and BALB/c) and equally in both male and female mice (FIG. 1d, FIGS. 8c-e).

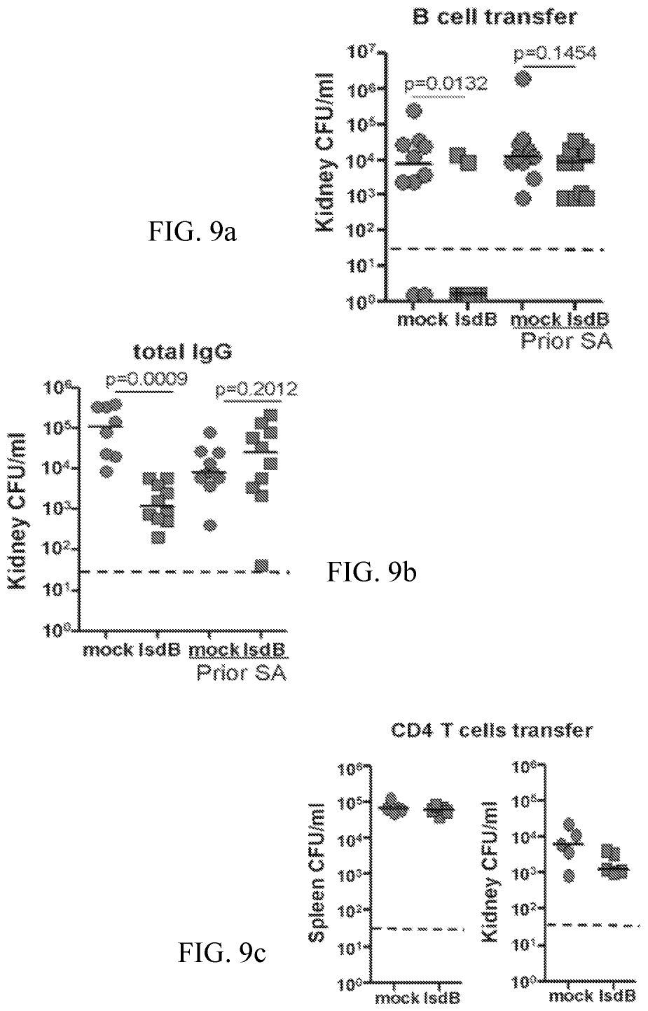

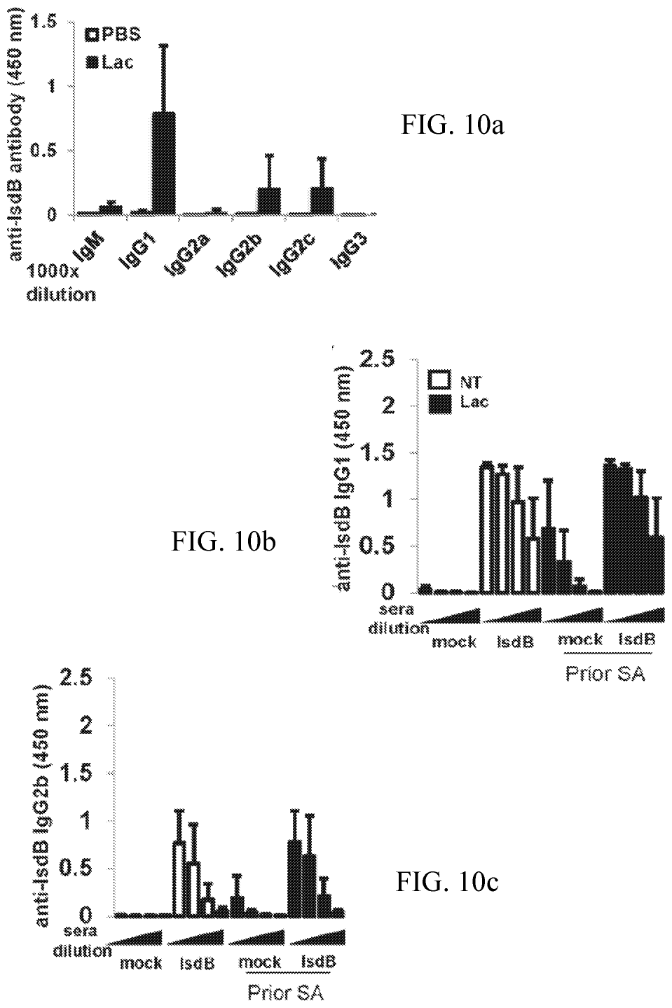

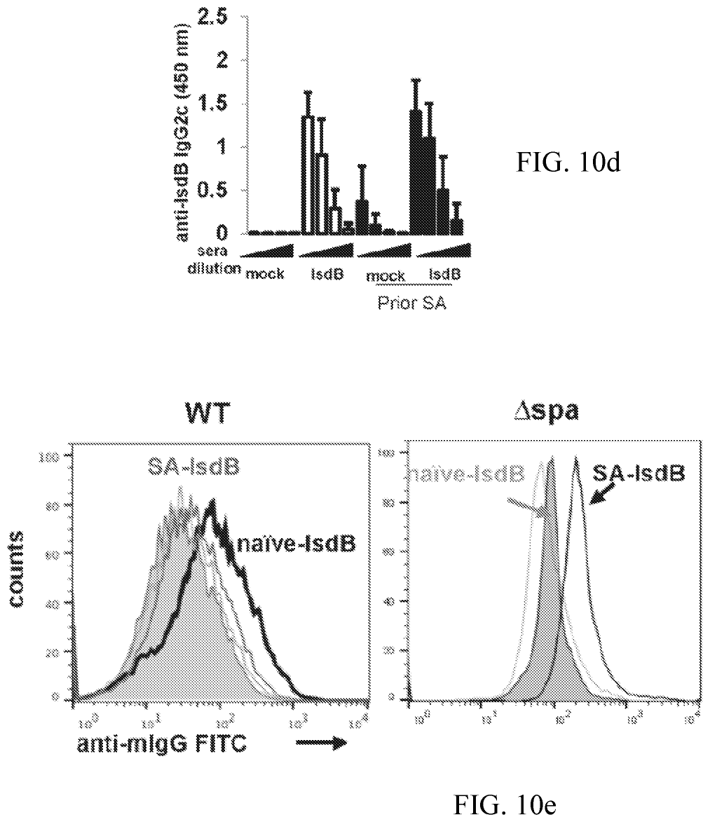

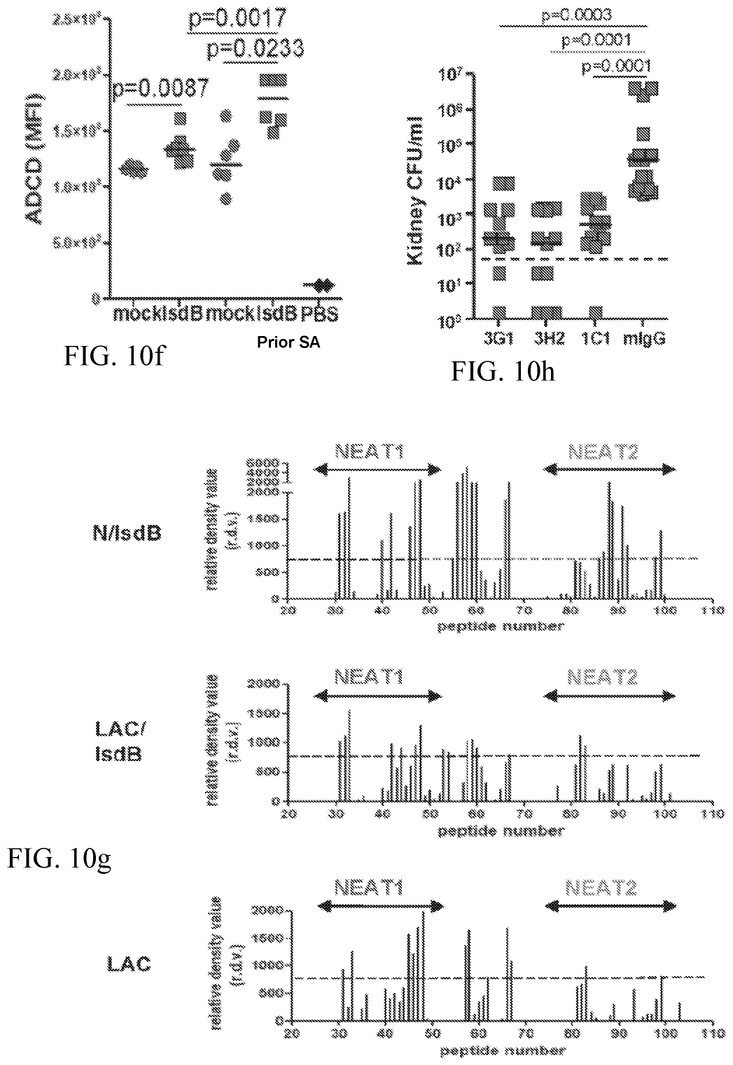

Vaccine suppression did not depend on innate training mechanisms. Adoptive transfer of B cells from SA-infected mice followed by vaccination of the recipients was sufficient to induce vaccine suppression (FIG. 9a). In addition, vaccine suppression persisted for at least 3 weeks from the time of last SA infection (FIG. 9b). Vaccine suppression was antigen-specific based on efficacy of the IsdB vaccine in mice previously infected with the IsdB/HarA mutant that does not induce IsdB cross-reactive Ab (FIG. 1f, FIG. 9c)¹⁴. In naïve mice, IsdB vaccination conferred anti-SA protection through B lymphocytes and Ab, but SA-infection prior to vaccination abrogated the ability of the B cells and total IgG to transfer anti-SA immunity (FIGS. 1g-h, FIGS. 10a-b). In comparison, transfer of CD4⁺ T cells from vaccinated SA-naïve mice was not protective (FIG. 10c). Hence, our data demonstrate that prior SA infection by different routes abrogated specific humoral immunity induced by IsdB vaccination.

Ineffective Antibody Responses Generated by S. aureus Infection and Subsequent IsdsB Vaccination

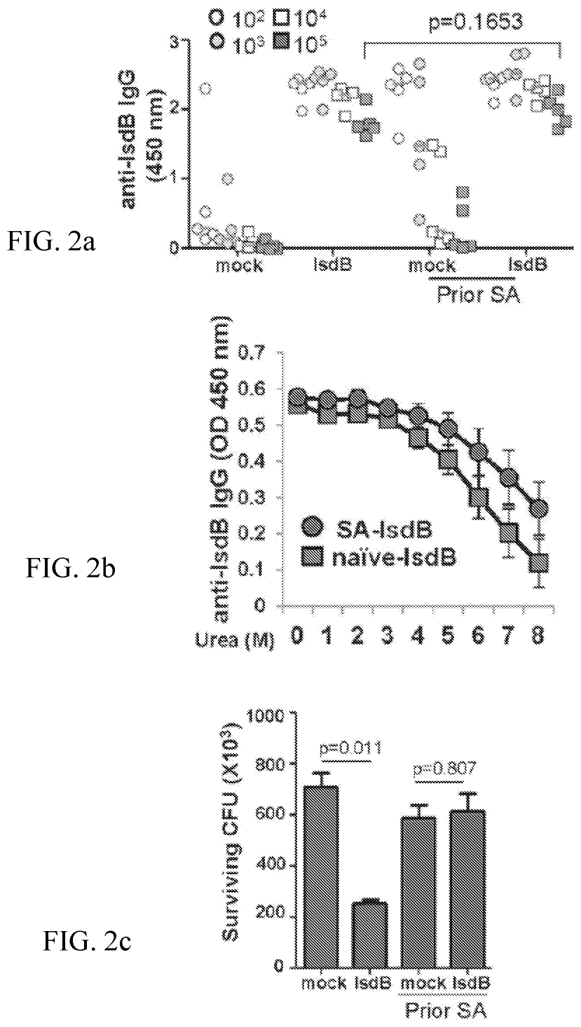

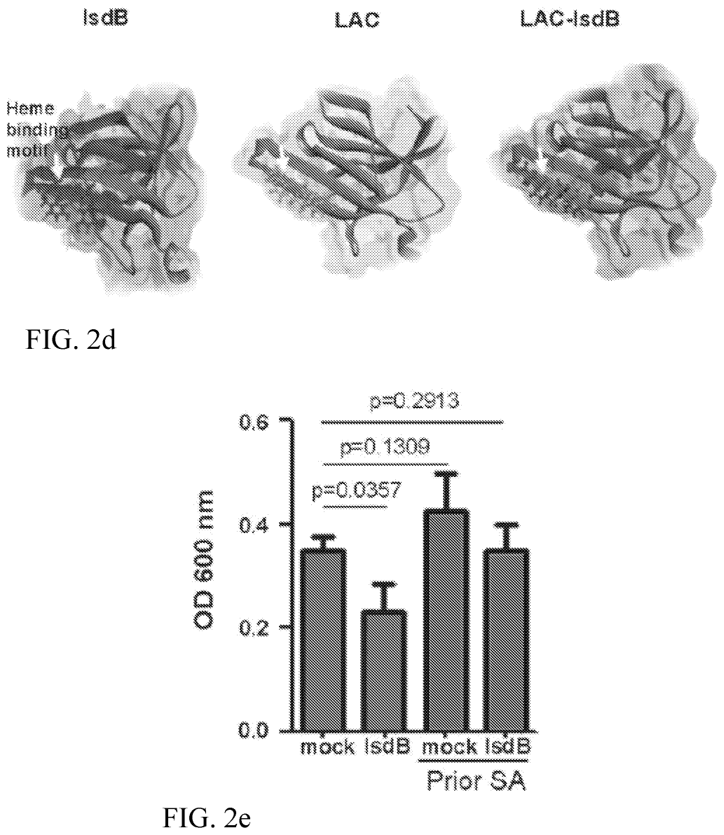

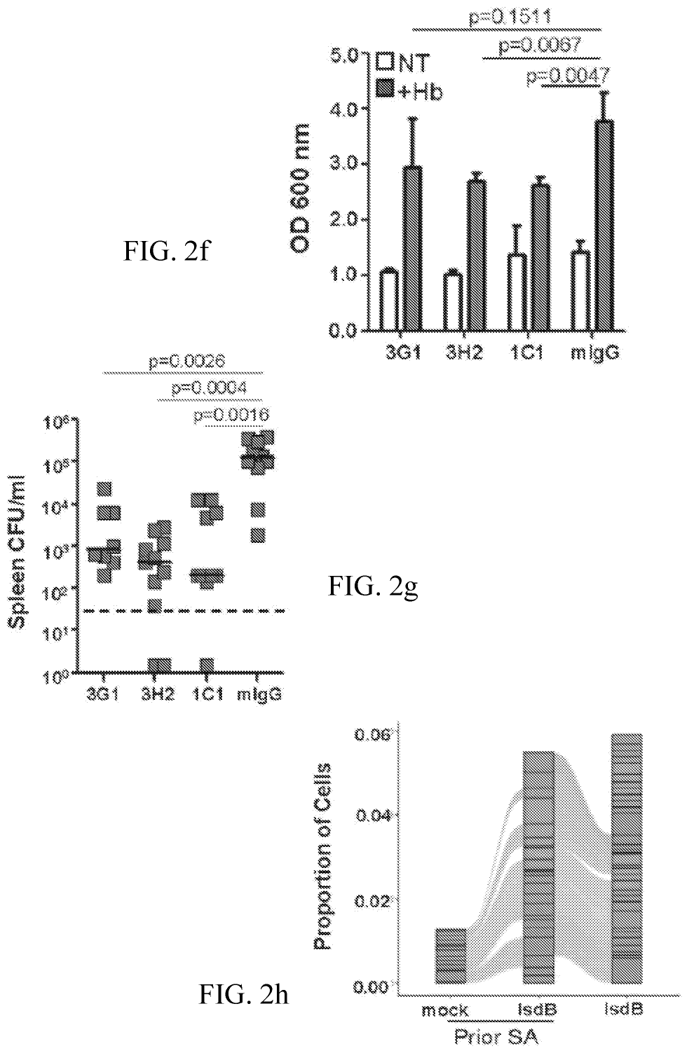

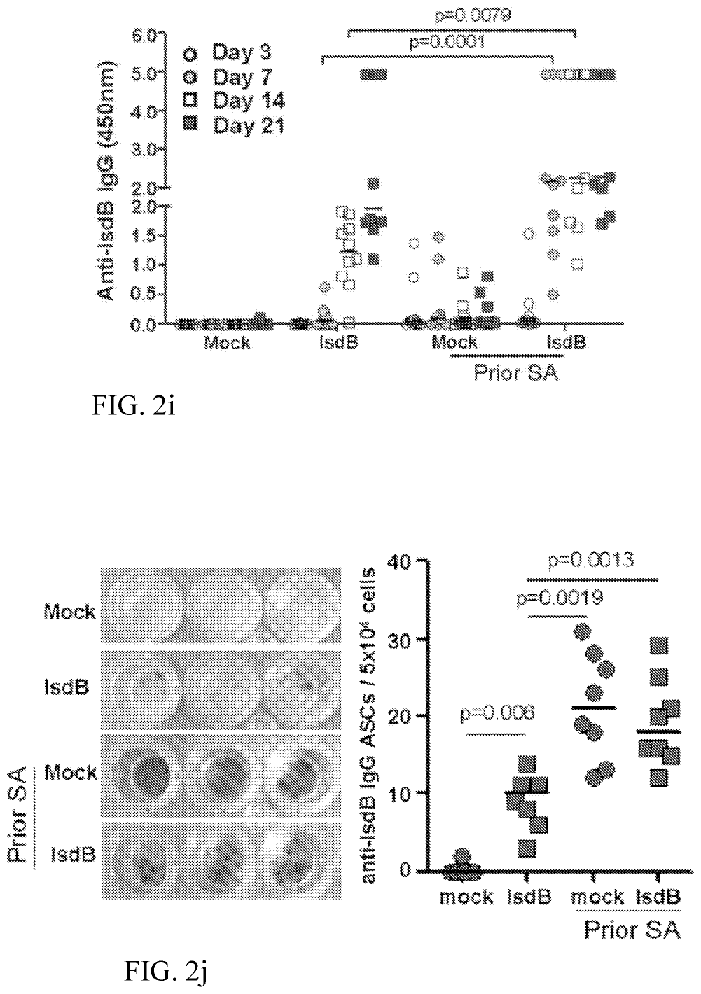

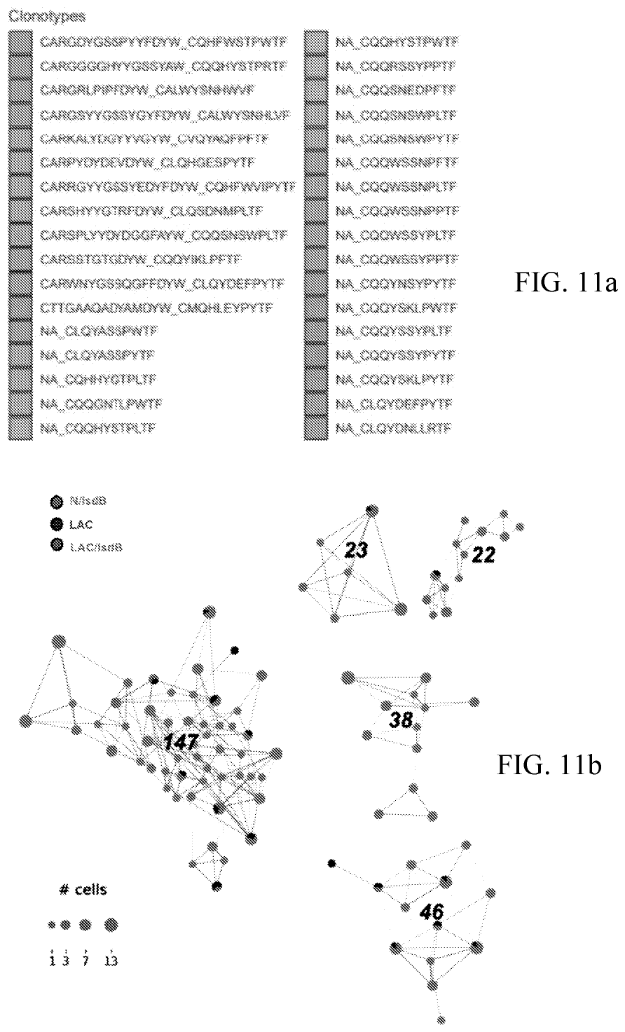

To determine why vaccine-generated Ab were ineffective in mice previously infected with SA, we compared IsdB-specific Ab generated by SA infection, by SA infection followed by IsdB vaccination, and by vaccination alone. We found that Ab titers, isotypes, affinity, and complement binding do not explain the differences in vaccine efficacy between naïve and pre-infected mice (FIGS. 2a-b, FIGS. 11 a-f). Analysis of Ab binding to an overlapping IsdB peptide array showed that Ab induced by SA infection or by vaccination in naïve and SA-infected mice mapped to different IsdB subdomains (FIG. 2d, FIG. 11g). Most notably, naïve mice immunized with IsdB generated abundant Ab that bound to a heme-binding domain of IsdB¹⁵. Ab to this region were infrequently induced after SA infection alone and moderately induced after IsdB vaccination of SA-infected mice (FIG. 11g). Corroborating these findings, a hemoglobin-dependent SA growth assay revealed that only Ab from naïve mice vaccinated with IsdB restricted growth of SA (FIG. 2e). Furthermore, three distinct monoclonal Ab (mAb) from a B cell hybridoma library, generated from naïve mice vaccinated with IsdB and selected based on restriction of growth of SA in the hemoglobin-dependent growth assay, were protective against SA infection in vivo (FIG. 2f-g, FIG. 11h).

In addition to differences in epitope map, we also found that opsonophagocytic killing of SA was more efficient with Ab generated by vaccination of naïve mice (FIG. 2c). Hence, we explored Ab glycosylation with a focus on sialylation which has been shown to inhibit opsonophagocytosis through blunting of Fc binding to FcrRIII and FcRIIb. We showed that IsdB-specific Ab generated by vaccination of SA-infected mice have enhanced α2,3 sialylation. Treatment of the Ab with α2,3 neuraminidase did not alter their binding to recombinant IsdB, but improved opsonophagocytic killing of S. aureus. Although binding of the Ab to FcRIIb improved with a-2,3 neuraminidase treatment, we did not observe a difference in FcRIIb binding by IsdB-specific Ab from naïve vaccinated versus SA-infected then vaccinated mice. These findings suggest α2,3 sialylation blunting of opsonophagocytosis through other Ab-receptor interactions on myeloid cells.

IsdB Vaccine Recalls the Ineffective Antibody Response in S. aureus-Infected Mice.

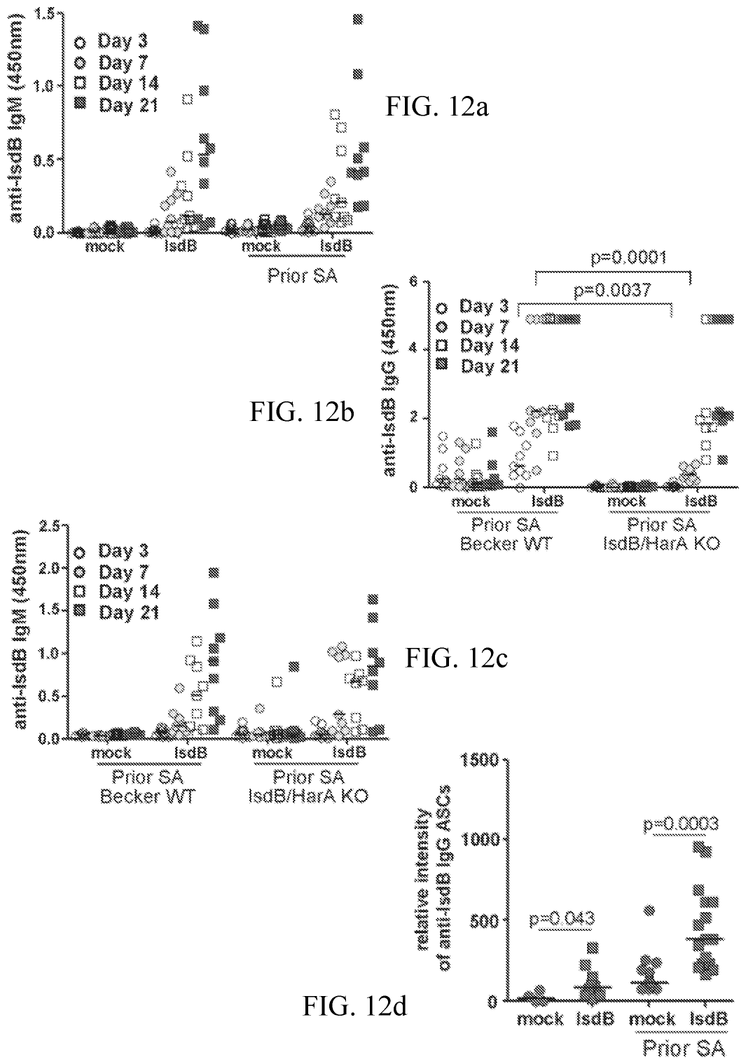

The findings of non-protective immunity conferred by both SA infection and SA infection with IsdB vaccination raised the possibility that IsdB vaccine recalls the ineffective IsdB-specific memory response if mice are pre-exposed to SA. To test this hypothesis, we analyzed the complementarity-determining region (CDR3) of IsdB-specific B cells and clonotypes associated with each infection and vaccination regimen. Administration of IsdB vaccine to previously infected mice resulted in the amplification of many IsdB-specific clonotypes found in SA-infected mice. IsdB vaccination of naïve mice generated some of the same clonotypes but produced a significant number of unique clonotypes (FIG. 2h, and FIG. 12), consistent with the results of epitope mapping using the IsdB peptide array. Moreover, we found that IsdB-specific IgG response increased sharply within 7 days of vaccinating pre-infected mice compared to vaccination of naïve mice (FIG. 2i, FIGS. 13a-c). The increased specific Ab levels and the number of activated Ab secreting cells isolated from mouse splenocytes on day 7 reflected the recall of the pre-existing humoral response (FIG. 2j, FIG. 13d). Overall, these findings suggest that, while IsdB vaccine in naïve mice induces a protective neutralizing Ab response to the heme-binding site, the same vaccine in pre-infected hosts recalls a non-protective response predominantly targeting epitopes away from the heme-binding site.

### Direct Antibody Competition Reduces Vaccine Efficacy but can be Overcome by Immunization Against IsdB NEAT2 Domain and Epitope.

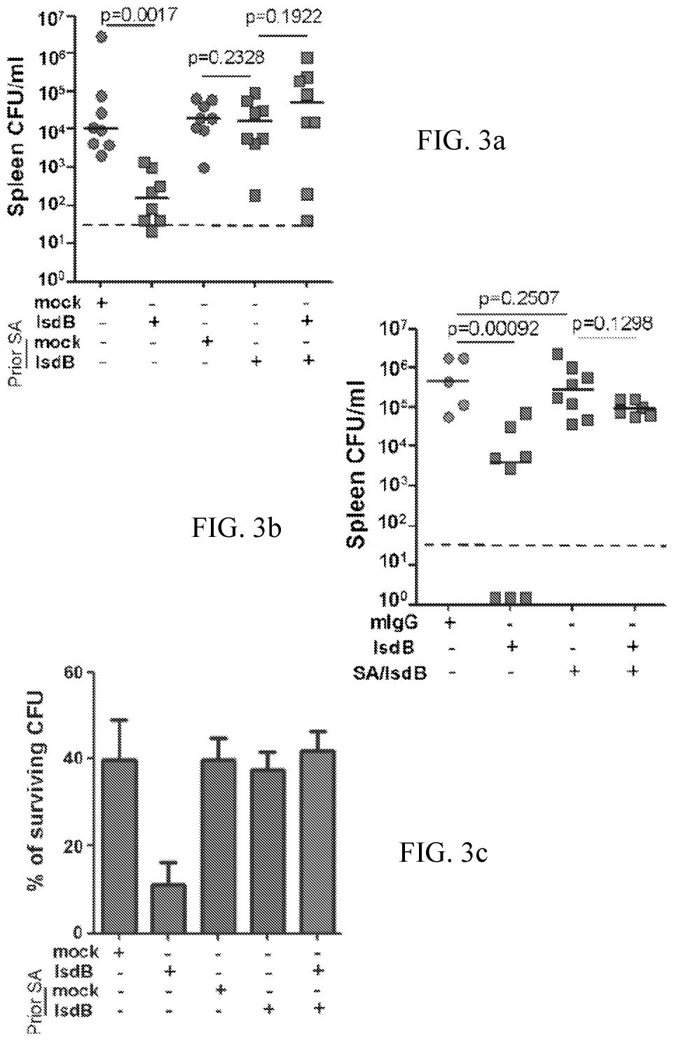

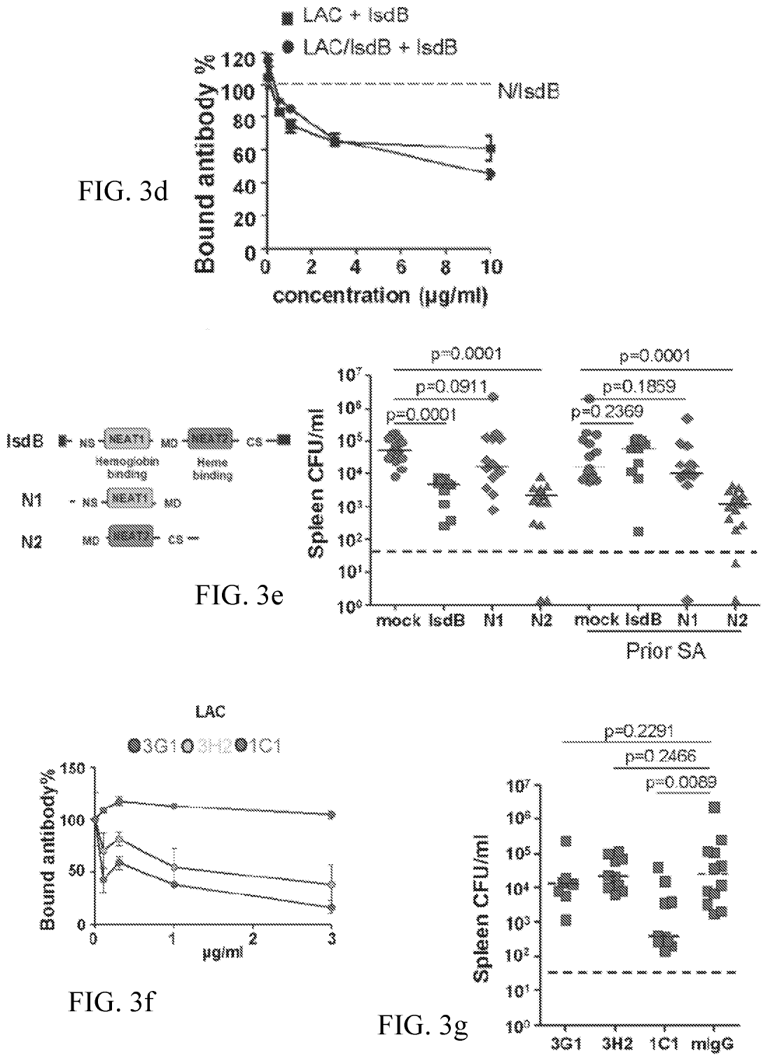

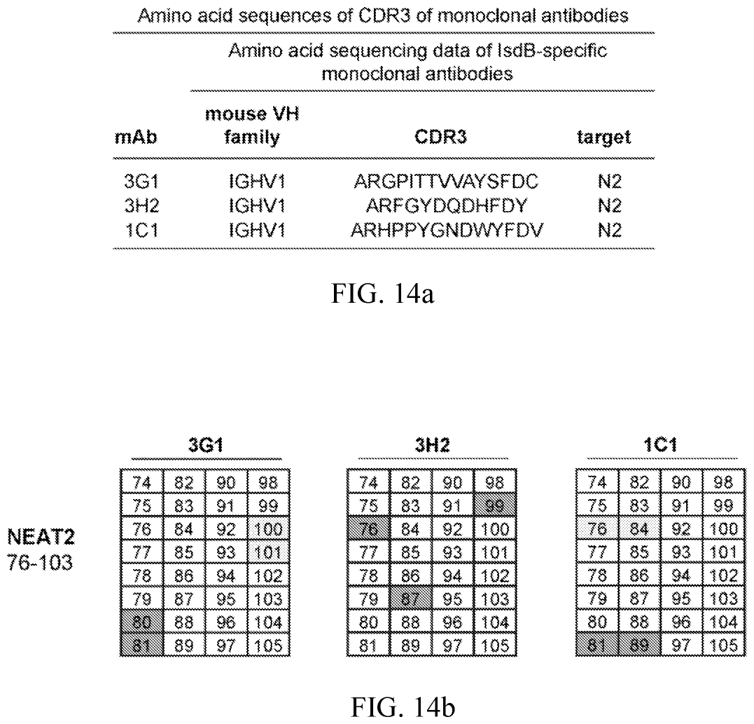

IsdB vaccine interference was remarkably effective even though recall of preexisting Ab responses limited but did not prevent de novo priming of IsdB-specific Ab responses to the heme-binding region (FIG. 2d). Because both protective and non-protective Ab target the same IsdB protein, we queried if recall of a non-protective Ab response could further blunt IsdB vaccine via antibody competition. In vivo, co-administration of non-protective sera (obtained from SA-infected then IsdB-vaccinated mice) abolished anti-SA protection conferred by sera from naïve IsdB-vaccinated mice (FIGS. 3a-b, FIGS. 14a-b). In vitro, co-administration of the non-protective Ab also inhibited protective Ab functions in opsonophagocytic killing and IsdB antigen binding assays (FIGS. 3c-d), although a 3-10-fold excess of non-protective specific-Ab was required to reduce binding by the protective Ab to the IsdB-coated plate.

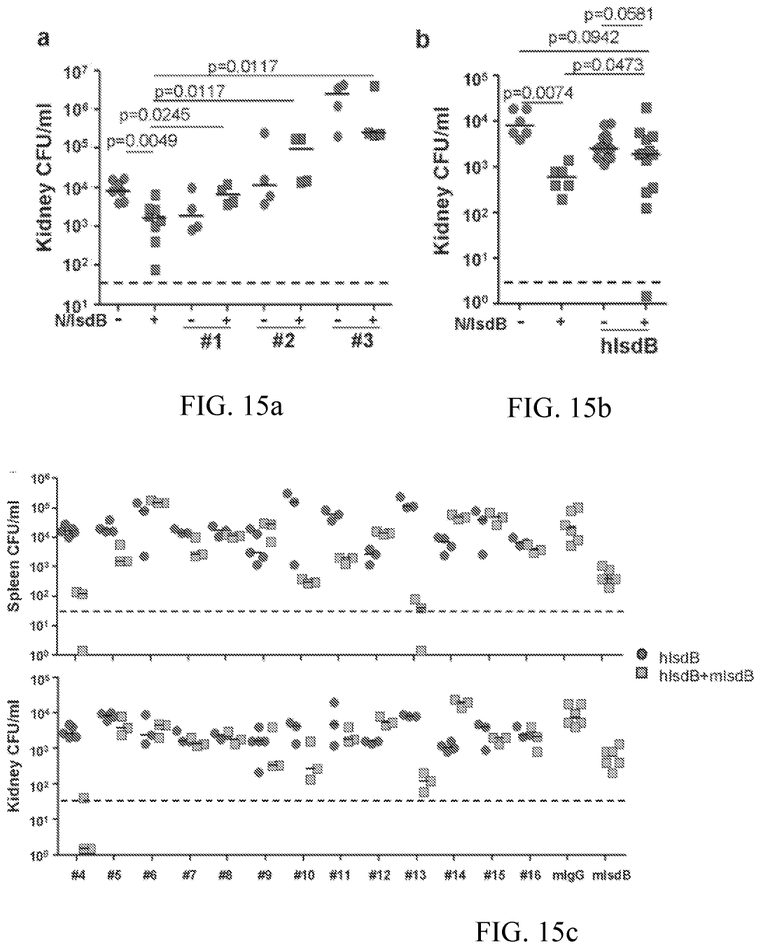

To determine if it is possible to overcome Ab suppression mediated by prior SA infection, we evaluated the effect of targeting the protective heme-binding NEAT2 domain of IsdB and the previously characterized NEAT2-targeting mAbs (FIGS. 2f-g, FIGS. 15a-b)¹⁵. Immunization of naïve mice with recombinant NEAT2, but not NEAT1, protected against SA challenge in naïve mice (FIG. 3e). Importantly, immunization with NEAT2 also overcame suppression by prior SA-infection (FIG. 3e). Conversely, only one of the three NEAT2 epitope-specific mAbs resisted competition by Ab induced by SA infection, both in vitro in a binding assay and in vivo (FIGS. 3f-g, FIGS. 14e). We conclude that despite vaccine interference, immunization against the NEAT2 subdomain or a selective epitope of IsdB induces protection in previously infected mice.

Non-Protective Human Anti-S. aureus Antibodies Reduce the Efficacy of IsdB and ClfA Passive Vaccines.

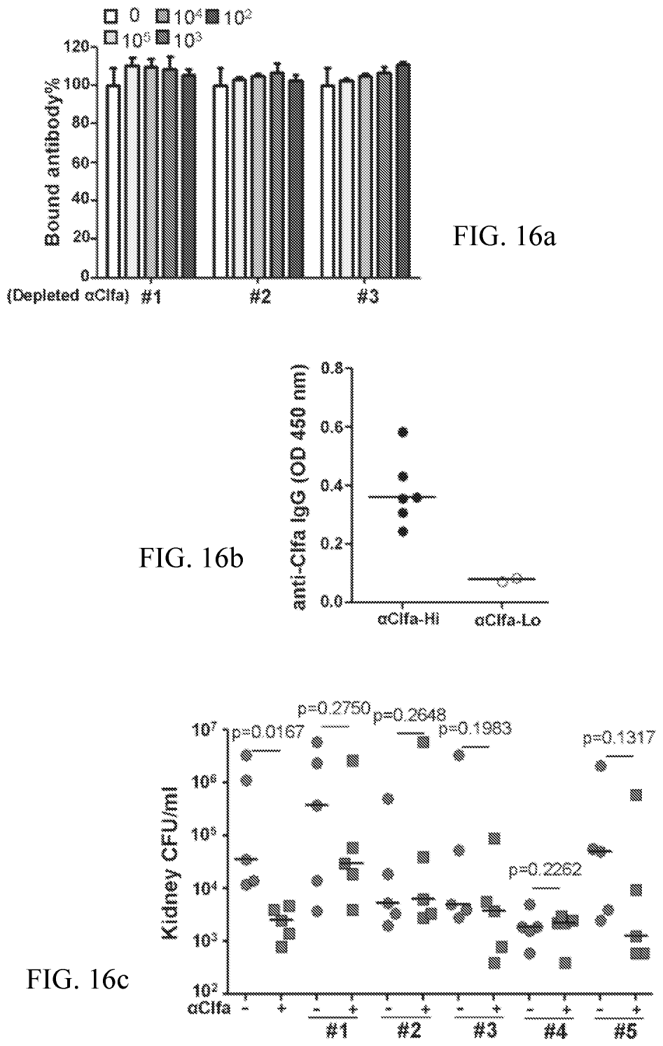

We next queried if pre-existing IsdB-specific Ab from adult humans can modify the protective function of IsdB-vaccine generated Ab. IsdB-specific Ab, which are abundant in human sera, decreased binding of vaccine-generated protective specific Ab to 60% at a 10:1 excess molar ratio (FIGS. 4a-b). To evaluate the in vivo suppressive functions of human Ab, we developed and validated an adoptive model of human Ab transfer and protection from SA challenge in mice¹⁶. In the model, we showed that both human sera and purified IsdB-specific Ab were non-protective against SA challenge. When co-administered with vaccine-generated protective mouse sera at near equal volumes, the sera blunted the anti-SA efficacy of the protective Ab (FIG. 4c, FIG. 16a).

To date, most failed staphylococcal vaccine trials have consisted of passive immunizations⁵, Simulating passive immunization in human hosts, we showed that IsdB-specific murine Ab lose their protective function when adoptively transferred to mice pre-infused with IsdB-specific human Ab (FIG. 4d, FIG. 16b-c). Extending our finding to the SA clumping factor (ClfA) vaccine that failed passive immunization trials^{17,18}, we showed that human sera with high anti-ClfA titers blocked ClfA binding by ClfA-vaccinated mouse sera, but binding was unaffected by ClfA-Ab depleted human sera (FIG. 4e, FIGS. 17a-b). In vivo, a human anti-ClfA mAb that failed clinical trial, Tefibazumab¹⁷, protected mice from SA infection when administered alone or after low ClfA Ab-titer human serum infusion but was ineffective when administered after (non-protective) high ClfA-titer human sera (FIG. 4f, FIG. 12c). Together, these findings support a suppressive role of pre-existing SA Ab in SA vaccine failures.

In conclusion, this study provides a mechanistic framework to explain the failure of the IsdB vaccine trial. SA infection, through yet unclear mechanisms, induces preferential generation of Ab to the non-neutralizing NEAT1 domain of IsdB, in contrast to IsdB vaccination in naïve mice which generates robust humoral response to the protective heme-binding NEAT2 domain. Additionally, SA infection enhances α2,3 sialylation of anti-IsdB antibodies which leads to blunting of SA killing by opsonophagocytosis.

When administered to mice previously infected with SA, the otherwise protective IsdB vaccine recalls the non-protective antibody response albeit permitting modest priming of Ab response to the heme-binding region. This study demonstrated that the recalled non-protective Ab response effectively blunted the protective vaccine response by competitive binding. These findings support recall of non-protective antibody response and antibody competition as two critical mechanisms that could explain the failure of active and passive staphylococcal vaccines.

Vaccine interference by pre-existing anti-SA immunity bears similarity to the concept of original antigenic sin that explains the decreased efficacy of influenza vaccines to influenza strains that have undergone seasonal drift¹⁹. Unlike the original concept where differences in antigenic targets exist between the initial and subsequent antigen exposure, SA IsdB vaccine is generated to a highly conserved antigen and is administered to human subjects with abundant pre-existing specific Ab in the failed trial. Therefore, any protective Ab generated needs to overcome competition from the potentially non-protective Ab. This is arguably a more robust mechanism of interference than that encountered by seasonal influenza vaccines. We showed that targeting of the heme-binding domain and select epitopes of IsdB can overcome this competition.

Unlike the original antigenic sin concept that explains reduced or even deleterious host responses after repeat infections with certain RNA viruses, e.g., influenza, HIV, RSV, and dengue virus, original antigenic sin pertinent to conserved IsdB may find broader application to other SA vaccines and to vaccines against the many pathogens that have adapted to a lifestyle of coexistence with the human host. Taken together, the integration of prior host-pathogen interaction into vaccine studies can help explain the failure of seemingly successful experimental SA vaccines that have been to date studied in silo in naïve animal models.

### EXAMPLES

Aspects of the present teachings may be further understood in light of the following examples, which should not be construed as limiting the scope of the present teachings in any way.

### Ethics Statement

Mouse studies were reviewed and approved by the Institutional Animal Care and Use Committee. Mouse experiments were conducted in accordance with recommendations listed in the Animal Care Program at University of California, San Diego and Cedars-Sinai Medical Center's regulations and recommendations on animal experiments. Experimentations using human blood were approved by the UCSD Human Research Protection Program. Prior informed consents were obtained from the human subjects. Experimental protocols were approved by the UCSD Biosafety Committee.

Murine Models of S. aureus Infection

C57BL/6, BALB/c, and CBA mice were purchased from Charles River Laboratories. S. aureus Becker and S. aureus Becker IsdB/HarA deletion mutant were gifts from Dr. Secore (Merck). Overnight S. aureus cultures were diluted 1:200 in Todd Hewitt broth (THB) and grown to an optical density of 0.8. Unless otherwise stated, 6-8 weeks old female mice were administered 2×10⁷ LAC (USA 300) i.p. for each S. aureus challenge. Spleen and kidneys were harvested 24 hr after the last infection, homogenized in phosphate-buffered saline (PBS) and plated on THB agar plates for CFU enumeration. Other S. aureus inocula were: 5×10⁷ LAC i.p. for the LD₉₀ infection, 2×10⁷ SA113 i.p., 4×10⁷ Newman or Becker (WT or IsdB/HarA mutant) i.p., 4×10⁷ LAC s.c., and 1×10⁶ LAC i.v.

### Cloning and Protein Expression

The IsdB gene was amplified from LAC using primers: 5′IsdB (5′-GGTCGCGGATCCAACAAACAGCAAAAAGAATTT-3′)(SEQ ID NO: 3) and 3′IsdB (5′-GGTGGTGCTCGAGTTTAGTTTTTACGTTTTCTAGGTAATAC-3′)(SEQ ID NO: 4). The PCR product was cloned into pET28 expression vector (Novagen) and expressed as described previously with some modifications (1). Briefly, IsdB-expressing plasmids were used to transform E. coli BL21 (DE3) cells (NEB) to produce a His-tagged protein with 1 mM of isopropyl-β-D-thiogalactoside (IPTG) for 2 hours. Recombinant E. coli was centrifuged and suspended in lysis buffer (50 mM Tris-HCl (pH 8.0), 0.1 M NaCl, 2 mM MgCl₂, 10 mM imidazole, 0.1% Tween 80, 1% Triton X100, PMSF, lysozyme (2 mg/ml). His-tagged IsdB was purified from the clarified lysate by His60 Ni Superflow Resin (Takara) chromatography. The column was washed with 20 mM Tris-HCl (pH 8.0), 150 mM NaCl, 0.1% Tween 80 and His-tagged IsdB was eluted with 300 mM imidazole, 20 mM Tris-HCl (pH 7.5), 150 mM NaCl and 0.1% Tween 80. To generate truncated constructs of IsdB containing single domain (FIG. 3 E), primers: NEAT1, 5′-CAAGCAGCAGCTGAAGAAACAGG-3′ (SEQ ID NO: 5) and 5′-TTA-TTTGAATTTATCTGCactgttataaattgg-3′ (SEQ ID NO: 6); NEAT2, 5′-ACTGAAGAAGATTATAAAGCTG-AAA-3′ (SEQ ID NO: 7) and 5′-TTAGTTTTTACGTTTTCTAGGT-3′ (SEQ ID NO: 8) were applied to PCR reactions using full length IsdB as template. Recombinant IsdB domains were purified as described above.

Immunization with IsdB Vaccine and Antibody Purification

Mice were immunized i.p. three times with IsdB (75 μg, 50 μg and 50 μg) plus aluminum hydroxide (alum, InvivoGen) (450 μg per dose) or with aluminum hydroxide alone at 7-day intervals. Mouse sera were screened for reactivity to IsdB by ELISA. IsdB-specific antibodies were purified from human or mouse sera using immobilized IsdB agarose columns (NHS-activated agarose, ThermoFisher Scientific).

### Opsonophagocytic Killing Assay

Opsonophagocytic killing assay (OPK) was performed as described²⁰. Mouse neutrophils were isolated from bone marrow by MojoSort™ Mouse Neutrophil Isolation Kit (BioLegend). Overnight culture of S. aureus LAC was diluted 1:200 in Todd Hewitt broth (THB) and grown to an optical density of 0.6. S. aureus was washed, resuspended in PBS, and incubated with mouse sera at 37° C. for 20 min, then added to 105 mouse neutrophils a multiplicity of infection (MOI) of 1:0.5 in the presence of 2% normal mouse serum. Following incubation at 37° C. for 1 hr with agitation at 200 rpm, samples were plated on THB agar plates for CFU enumeration.

### Antibody Avidity Assay

Immunized serums were diluted 1:1000 in PBS containing 1% BSA and added to 96-well plates coated with recombinant IsdB (1 μg/ml). After incubation for 1 hr, different concentration of urea (0 to 8 M) with 0.05% Tween 20 in PBS were treated for 15 mins. Plates were washed and bound antibodies were detected by horseradish peroxidase (HRP)-conjugated goat anti-mouse IgG (BioLegend).

### Antibody-Dependent Complement Deposition Assay (ADCD)

Antibody-dependent complement deposition (ADCD) was described previously²¹. Briefly, biotinylated IsdB (EZ-Link™ Sulfo-NHS-Biotin, ThermoFisher Scientific) was coupled to red fluorescence NeutrAvidin beads (ThermoFisher Scientific). Antigen coupled beads were washed with 0.05% Tween20 in PBS and incubated with 10 μl of immunized serums (1:10 dilution) in 0.1% BSA for 2 hrs at 37° C. Antibodies-beads complex were washed and incubated with 100 μl of complement factors from guinea pig (MP Biomedicals) for 20 mins at 37° C. and washed with 15 mM EDTA/PBS to stop complement reaction. C3 deposition was detected by FITC-conjugated anti-C3 polyclonal antibody (1:100 dilution, MP Biomedicals) and subjected to flow analysis.

### IsdB ELISA and ELISPOT Assay

IsdB-specific antibody levels in human and mouse sera were measured by ELISA as described previously¹⁴. Briefly, sera were serially diluted in PBS containing 1% BSA and added to 96-well plates coated with recombinant IsdB (1 μg/ml). Bound antibodies were detected by horseradish peroxidase (HRP)-conjugated goat anti-mouse IgG or anti-mouse IgM (BioLegend).

ELISPOT assay was performed on splenocytes isolated from IsdB-immunized mice to quantify IsdB-specific IgG-secreting cells. Splenocytes were serially diluted in RPMI containing 10% FBS from 5×10⁶ cells/well into 96-well PVDF plate (Corning) coated with recombinant IsdB (1 μg/well) and incubated for 6 hr at 37° C. Afterwards, the cells were detached using PBS with 0.1% Tween20 and the plates were incubated for an hour at 25° C. with alkaline phosphatase (AP)-conjugated anti-mouse IgG (Southern biotech) and developed with BCIP/NBT solution (MABTECH). Spots were analyzed using ImageJ.

Hemoglobin-Dependent S. aureus Growth Assay

Hemoglobin-dependent growth assay was performed as described previously¹⁵. Briefly, S. aureus LAC was grown in RPMI medium containing 0.1 Casamino Acids and 200 M 2,2′ Bipyridyl. Overnight cultures were washed twice in RPMI containing 500 M 2,2′ Bipyridyl and diluted 1:100 in 200 μl of RPMI medium containing 500 M 2,2′ bipyridyl, 25 M ZnCl₂, 25 μM MnCl₂, 1 mM MgCl₂, 100 μM CaCl₂), and antibodies or sera with or without 1 μM hemoglobin (Sigma). Bacterial growth at 37° C. as measured by OD₆₀₀ and was recorded using a Perkin Elmer Ensire Alpha plate reader.

Flow Cytometric Analysis of IsdB Antibody Binding to S. aureus

For measurement of IsdB antibody binding to S. aureus cell surface, 1×10⁷ LAC were washed and incubated with 10 μl of immunized serum, then FITC-conjugated anti-mouse IgG. Fluorescence intensity was analyzed by FACSCanto (BD Biosciences) and FlowJo software.

### Epitope Mapping Using an Overlapping IsdB Peptide Array

The IsdB-binding profile of serum antibodies induced by IsdB immunization in naïve and SA-experienced mice was evaluated using a continuous peptide array composed of 141 15-mer peptides with an 11-residue overlap, covering the full-length IsdB. Peptides were printed on immobilized PVDF membranes. Membranes were blocked with TBST containing 1% BSA, then pooled serum samples were diluted 1:1000 in blocking solution and added to the peptide array. Bound antibodies were detected by incubation with 800CW goat anti-mouse IgG (LI-COR) and fluorescent intensity was determined by Odyssey Imager (LI-COR). For epitope mapping of mAb 3G1, 3H2 and 1C1, 10 μg/ml of mAb were diluted in blocking buffer and incubated in peptide array of NEAT2 domain (#76-#103) for 1 hr. Bound antibodies were detected by horseradish peroxidase (HRP)-conjugated goat anti-mouse IgG.

### Adoptive Transfer of T Lymphocytes, B Lymphocytes, Sera or Purified Antibodies

Splenic CD4⁺ T cells or CD19⁺ B cells were isolated using kits following instructions provided by the manufacturer (MojoSort™, Biolegend). 2×10⁷ T cells or B cells were injected i.v. into recipient mice. IsdB or ClfA immune sera were generated by SA-infection or immunization as described above. Human sera were obtained from anonymized adult human volunteers. IsdB-specific antibodies were purified from human or mouse sera using immobilized IsdB agarose columns (NHS-activated agarose, ThermoFisher Scientific). Tefibazumab, a monoclonal Antibody against ClfA used in clinical trials²² was purchased from Creative Biolabs (TAB-029). Immunized sera or purified antibodies were diluted in PBS and injected i.v. (sera) or i.p. (purified antibodies) into recipient mice.

### Generation of Anti-IsdB Monoclonal Antibodies

Monoclonal antibodies were generated as previous described¹⁴. Briefly, mice were immunized i.p. 3 times with 75 μg IsdB formulated with aluminum hydroxide. 20 μg IsdB were injected i.p. 3 days prior to cell fusion. Splenocytes were fused with mouse myeloma partner P3X (ATCC CRL-1580) by using polyethylene glycol 1500 (Sigma) at a ratio of 3:1. The fused cells were plated and screened by ELISA for reactivity to IsdB as previously described. The complementarity-determining region 3 (CDR3) were analyzed as previously described²³.

### BCR Genotyping

The BCR sequence data were processed using the Immcantation toolbox (v4.0.0) with default parameter values. Initial germline V(D)J gene annotation was performed using IgBLAST²⁴ with IMGT germline sequence databases²⁵. The IgBLAST database was further used to assign V(D)J gene annotations to the BCR FASTA files for each sample using the Change-O package²⁶. The derived matrix contains sequence alignment information for each sample with both light and heavy chain sequences, and individualized genotypes were inferred using the TIgGER package²′ and used to finalize V(D)J annotations.

### Preparation of Single-Cell Sequencing

Splenocytes from IsdB immunized or LAC-experienced mice were incubated with phycoerythrin (PE)-labeled IsdB and allophycocyanin-conjugated anti-mouse CD45R/B220. IsdB+B220+ cells were sorted by FACSAria II (BD) and subjected to single-cell preparation by using a Single Cell 5′ Library and Gel Bead kit and Chromium Single Cell A Chip kit, the cell suspension was loaded onto a Chromium single cell controller to generate single-cell gel beads in the emulsion (GEMs) according to the manufacturer's protocol (10× Genomics). scRNA-seq libraries were constructed using a Chromium Single Cell V(D)J Enrichment Kit, Mouse B Cell following instructions provided by the manufacturer (10× Genomics). The libraries were sequenced using an Illumina Novaseq6000 sequencer with a paired-end 150-bp (PE150) reading strategy (performed by Institute for Genomic Medicine, UCSD).

### Identification of Clones and Comparisons of Clonotypes

The clonal groups were identified by the R package—scRepertoire²⁸ based on paired heavy and light chains. To determine clonal groups, we first used the filtered contig annotation obtained from the results performed using the Cell Ranger Single-Cell Software Suites (http://software.10xgenomics.com/single-cell/overview/welcome). Then, for the cells with high quality paired heavy and light chains were sequenced, clones were assigned based on strict definition of clonotype using the CTstrict( ) function that considers clonally related two sequences with identical V gene usage and >85% normalized Levenshtein distance of the nucleotide sequence. The clonotype changes between samples were visualized by the compareClonotypes( ) function with the clones called by amino acid sequence of the CDR3 region.

### Clonal Lineage Analysis (CDR3 Neighborhood Network)

To indicate convergent BCR evolution, we further used Scirpy²⁹ to identify clonotype clusters based on CDR3 amino acid sequence similarity. An identified clonotype cluster is a higher-order aggregation of clonotypes that have different CDR3 nucleotide sequences, but might recognize the same antigen because they have the same or similar CDR3 amino acid sequence. Specifically, the clonotype clusters were identified by leveraging the Parasail library³⁰ to compute pairwise sequence alignments and identify clusters of B cells. The clonotype neighborhood networks was then visualized using Scirpy that makes use of the sparse-matrix implementation from the scipy package³¹.

### Statistics

Data are expressed as mean and were analyzed using Student's t test (two-tailed tests). In vivo experiments were analyzed using non-parametric Mann-Whitney U-test or Kruskal-Wallis test in the case of missing normality. Log rank test was used for analysis of mouse survival. Analyses were performed using GraphPad Prism 5. p values of <0.05 were considered to be statistically significant.

### Other Embodiments

The detailed description set-forth above is provided to aid those skilled in the art in practicing the present invention. However, the invention described and claimed herein is not to be limited in scope by the specific embodiments herein disclosed because these embodiments are intended as illustration of several aspects of the invention. Any equivalent embodiments are intended to be within the scope of this invention. Indeed, various modifications of the invention in addition to those shown and described herein will become apparent to those skilled in the art from the foregoing description which do not depart from the spirit or scope of the present inventive discovery. Such modifications are also intended to fall within the scope of the appended claims.

### REFERENCES CITED

All publications, patents, patent applications and other references cited in this application are incorporated herein by reference in their entirety for all purposes to the same extent as if each individual publication, patent, patent application or other reference was specifically and individually indicated to be incorporated by reference in its entirety for all purposes. Citation of a reference herein shall not be construed as an admission that such is prior art to the present invention.

Specifically intended to be within the scope of the present invention, and incorporated herein by reference in its entirety, is the following publication: Tsai et al., “Non-protective immune imprint underlies failure of S. aureus IsdB vaccine” Cell Host & Microbe 30, 1163-1172 (2022).

Other publications incorporated herein by reference in their entirety include:
1 Wright, A. E. Notes on the Treatment of Furunculosis, Sycosis, and Acne by the Inoculation of a Staphylococcus aureus Vaccine. Lancet, 874-884 (1902).
2 Miller, L. S., Fowler, V. G., Shukla, S. K., Rose, W. E. & Proctor, R. A. Development of a vaccine against Staphylococcus aureus invasive infections: Evidence based on human immunity, genetics and bacterial evasion mechanisms. FEMS Microbiol Rev 44, 123-153, doi:10.1093/femsre/fuz030 (2020).
3 Lebon, A. et al. Dynamics and determinants of Staphylococcus aureus carriage in infancy: the Generation R Study. J Clin Microbiol 46, 3517-3521, doi:10.1128/JCM.00641-08 (2008).
4 Kluytmans, J., van Belkum, A. & Verbrugh, H. Nasal carriage of Staphylococcus aureus: epidemiology, underlying mechanisms, and associated risks. Clin Microbiol Rev 10, 505-520 (1997).
5 Fowler, V. G., Jr. & Proctor, R. A. Where does a Staphylococcus aureus vaccine stand? Clin Microbiol Infect 20 Suppl 5, 66-75, doi:10.1111/1469-0691.12570 (2014).
6 Goodyear, C. S. & Silverman, G. J. Death by a B cell superantigen: In vivo VH-targeted apoptotic supraclonal B cell deletion by a Staphylococcal Toxin. J Exp Med 197, 1125-1139, doi:10.1084/jem.20020552 (2003).
7 Alonzo, F., 3rd et al. CCR5 is a receptor for Staphylococcus aureus leukotoxin ED. Nature 493, 51-55, doi:10.1038/nature11724 (2013).
8 Sanchez, M. et al. O-Acetylation of Peptidoglycan Limits Helper T Cell Priming and Permits Staphylococcus aureus Reinfection. Cell Host Microbe 22, 543-551 e544, doi:10.1016/j.chom.2017.08.008 (2017).
9 Kappler, J. et al. V beta-specific stimulation of human T cells by staphylococcal toxins. Science 244, 811-813, doi:10.1126/science.2524876 (1989).
10 Gerlach, D. et al. Methicillin-resistant Staphylococcus aureus alters cell wall glycosylation to evade immunity. Nature 563, 705-709, doi:10.1038/s41586-018-0730-x (2018).
11 Fowler, V. G. et al. Effect of an investigational vaccine for preventing Staphylococcus aureus infections after cardiothoracic surgery: a randomized trial. JAMA 309, 1368-1378, doi:10.1001/jama.2013.3010 (2013).
12 Zorman, J. K. et al. Naturally occurring IgG antibody levels to the Staphylococcus aureus protein IsdB in humans. Hum Vaccin Immunother 9, 1857-1864, doi:10.4161/hv.25253 (2013).
13 Stranger-Jones, Y. K., Bae, T. & Schneewind, O. Vaccine assembly from surface proteins of Staphylococcus aureus. Proc Natl Acad Sci USA 103, 16942-16947, doi:10.1073/pnas.0606863103 (2006).
14 Kuklin, N. A. et al. A novel Staphylococcus aureus vaccine: iron surface determinant B induces rapid antibody responses in rhesus macaques and specific increased survival in a murine S. aureus sepsis model. Infect Immun 74, 2215-2223, doi:10.1128/IAI.74.4.2215-2223.2006 (2006).
15 Bennett, M. R. et al. Human VH1-69 Gene-Encoded Human Monoclonal Antibodies against Staphylococcus aureus IsdB Use at Least Three Distinct Modes of Binding To Inhibit Bacterial Growth and Pathogenesis. mBio 10, doi:10.1128/mBio.02473-19 (2019).
16 Tsai, C. M. et al. Adoptive Transfer of Serum Samples From Children With Invasive Staphylococcal Infection and Protection Against Staphylococcus aureus Sepsis. J Infect Dis 223, 1222-1231, doi:10.1093/infdis/jiaa482 (2021).
17 Weems, J. J., Jr. et al. Phase II, randomized, double-blind, multicenter study comparing the safety and pharmacokinetics of tefibazumab to placebo for treatment of Staphylococcus aureus bacteremia. Antimicrob Agents Chemother 50, 2751-2755, doi:10.1128/AAC.00096-06 (2006).
18 DeJonge, M. et al. Clinical trial of safety and efficacy of INH-A21 for the prevention of nosocomial staphylococcal bloodstream infection in premature infants. J Pediatr 151, 260-265, 265 e261, doi:10.1016/j.jpeds.2007.04.060 (2007).
19 Francis, T. On the doctrine of original antigenic sin. Proc Am Philos Soc., 572-578 (1960).
20 Thomer, L. et al. Antibodies against a secreted product of Staphylococcus aureus trigger phagocytic killing. J Exp Med 213, 293-301, doi:10.1084/jem.20150074 (2016).
21 Fischinger, S. et al. A high-throughput, bead-based, antigen-specific assay to assess the ability of antibodies to induce complement activation. J Immunol Methods 473, 112630, doi:10.1016/j.jim.2019.07.002 (2019).
22 Hall, A. E. et al. Characterization of a protective monoclonal antibody recognizing Staphylococcus aureus MSCRAMM protein clumping factor A. Infect Immun 71, 6864-6870, doi:10.1128/IAI.71.12.6864-6870.2003 (2003).
23 Tiller, T., Busse, C. E. & Wardemann, H. Cloning and expression of murine Ig genes from single B cells. J Immunol Methods 350, 183-193, doi:10.1016/j.jim.2009.08.009 (2009).
24 Ye, J., Ma, N., Madden, T. L. & Ostell, J. M. IgBLAST: an immunoglobulin variable domain sequence analysis tool. Nucleic Acids Res 41, W34-40, doi:10.1093/nar/gkt382 (2013).
25 Giudicelli, V., Chaume, D. & Lefranc, M. P. IMGT/GENE-DB: a comprehensive database for human and mouse immunoglobulin and T cell receptor genes. Nucleic Acids Res 33, D256-261, doi:10.1093/nar/gki010 (2005).
26 Gupta, N. T. et al. Change-O: a toolkit for analyzing large-scale B cell immunoglobulin repertoire sequencing data. Bioinformatics 31, 3356-3358, doi:10.1093/bioinformatics/btv359 (2015).
27 Gadala-Maria, D., Yaari, G., Uduman, M. & Kleinstein, S. H. Automated analysis of high-throughput B-cell sequencing data reveals a high frequency of novel immunoglobulin V gene segment alleles. Proc Natl Acad Sci USA 112, E862-870, doi:10.1073/pnas. 1417683112 (2015).
28 Borcherding, N., Bormann, N. L. & Kraus, G. scRepertoire: An R-based toolkit for single-cell immune receptor analysis. F1000Res 9, 47, doi:10.12688/f1000research.22139.2 (2020).
29 Sturm, G. et al. Scirpy: a Scanpy extension for analyzing single-cell T-cell receptor-sequencing data. Bioinformatics 36, 4817-4818, doi:10.1093/bioinformatics/btaa611(2020).
30 Daily, J. Parasail: SIMD C library for global, semi-global, and local pairwise sequence alignments. BMC Bioinformatics 17, 81, doi:10.1186/s12859-016-0930-z (2016).
31 Virtanen, P. et al. SciPy 1.0: fundamental algorithms for scientific computing in Python. Nat Methods 17, 261-272, doi:10.1038/s41592-019-0686-2 (2020).

1. A vaccine composition comprising:
a NEAT2 polypeptide comprising an amino acid sequence corresponding to SEQ ID NO: 1, or variant or fragment thereof.

2. The vaccine composition of claim 1, further comprising a pharmaceutical carrier.

3. The vaccine composition of claim 1, wherein the variant comprises greater than 95% sequence identity to SEQ ID NO: 1.

4. The vaccine composition of claim 1, wherein the fragment comprises greater than 50 amino acids.

5-7. (canceled)

8. The vaccine composition of claim 1 further comprising IsdB vaccine selected from the group consisting of STAPHVAX manufactured by NABI, ALTASTAPH manufactured by NABI, PENTASTAPH manufactured by NABI/GSK, AUROGRAB manufactured by Novartis, VERONATE manufactured by Inhibitex, Tefibazumab manufactured by Inhibitex, Pagibaximab manufactured by Biosynexus, V710 manufactured by Merck, SAR279356 manufactured by Sanofi, NVD3 manufactured by Novadigm, STEBVAX manufactured by IBT, SA3Ag manufactured by Pfizer, PF-06290510 manufactured by Pfizer, and MEDI4893 manufactured by Medimmune.

9. A method of treating a subject having a S. aureus infection, the method comprising:
administering the vaccine composition of claim 1 to the subject, the administration resulting in immunogenicity.

10. A method of preventing S. aureus infection in a subject, the method comprising:
administering the vaccine composition of claim 1 to the subject, the administration resulting in immunogenicity.

11. A method for enhancing the effect of IsdB vaccine administration, the method comprising:
first immunizing a human against S. aureus infection with an IsdB vaccine followed by immunization of the human with the vaccine composition of claim 1, then followed by immunization of the human with the IsdB vaccine.

12. The method of claim 11, wherein the IsdB vaccine is selected from the group consisting of STAPHVAX manufactured by NABI, ALTASTAPH manufactured by NABI, PENTASTAPH manufactured by NABI/GSK, AUROGRAB manufactured by Novartis, VERONATE manufactured by Inhibitex, Tefibazumab manufactured by Inhibitex, Pagibaximab manufactured by Biosynexus, V710 manufactured by Merck, SAR279356 manufactured by Sanofi, NVD3 manufactured by Novadigm, STEBVAX manufactured by IBT, SA3Ag manufactured by Pfizer, PF-06290510 manufactured by Pfizer, and MEDI4893 manufactured by Medimmune.


SEQUENCE LISTING (8 sequences)

SEQ ID NO: 1 - protein, 377 aa, Staphylococcus aureus
TEEDYKAEKLLAPYKKAKTLERQVYELNKIQDKLPEKLKAEYKKKLEDTKKALDEQVKSAITEFQNVQPTNEKMTDLQDTKYVVYESVENNESMMDTFVKHPIKTGMLNGKKYMVMETTNDDYWKDFMVEGQRVRTISKDAKNNTRTIIFPYVEGKTLYDAIVKVHVKTIDYDGQYHVRIVDKEAFTKANTDKSNKKEQQDNSAKKEATPATPSKPTPSPVEKESQKQDSQKDDNKQLPSVEKENDASSESGKDKTPATKPTKGEVESSSTTPTKVVSTTQNVAKPTTASSKTTKDVVQTSAGSSEAKDSAPLQKANIKNTNDGHTQSQNNKNTQENKAKSLPQTGEESNKDMTLPLMALLALSSIVAFVLPRKRKN

SEQ ID NO: 2 - DNA, 1134 bp, Staphylococcus aureus
  source 1..1134: mol_type = other DNA
actgaagaagattataaagctgaaaaattattagcgccatataaaaaagcgaaaacactagaaagacaagtttatgaattaaataaaattcaagataaacttcctgaaaaattaaaggctgagtacaagaagaaattagaggatacaaagaaagctttagatgagcaagtgaaatcagctattactgaattccaaaatgtacaaccaacaaatgaaaaaatgactgatttacaagatacaaaatatgttgtttatgaaagtgttgagaataacgaatctatgatggatacttttgttaaacaccctattaaaacaggtatgcttaacggcaaaaaatatatggtcatggaaactactaatgacgattactggaaagatttcatggttgaaggtcaacgtgttagaactataagcaaagatgctaaaaataatactagaacaattattttcccatatgttgaaggtaaaactctatatgatgctatcgttaaagttcacgtaaaaacgattgattatgatggacaataccatgtcagaatcgttgataaagaagcatttacaaaagccaataccgataaatctaacaaaaaagaacaacaagataactcagctaagaaggaagctactccagctacgcctagcaaaccaacaccatcacctgttgaaaaagaatcacaaaaacaagacagccaaaaagatgacaataaacaattaccaagtgttgaaaaagaaaatgacgcatctagtgagtcaggtaaagacaaaacgcctgctacaaaaccaactaaaggtgaagtagaatcaagtagtacaactccaactaaggtagtatctacgactcaaaatgttgcaaaaccaacaactgcttcatcaaaaacaacaaaagatgttgttcaaacttcagcaggttctagcgaagcaaaagatagtgctccattacaaaaagcaaacattaaaaacacaaatgatggacacactcaaagccaaaacaataaaaatacacaagaaaataaagcaaaatcattaccacaaactggtgaagaatcaaataaagatatgacattaccattaatggcattattagctttaagtagcatcgttgcattcgtattacctagaaaacgtaaaaactaa

SEQ ID NO: 3 - DNA, 33 bp, Staphylococcus aureus
  source 1..33: mol_type = other DNA
ggtcgcggatccaacaaacagcaaaaagaattt

SEQ ID NO: 4 - DNA, 41 bp, Staphylococcus aureus
  source 1..41: mol_type = other DNA
ggtggtgctcgagtttagtttttacgttttctaggtaatac

SEQ ID NO: 5 - DNA, 23 bp, Staphylococcus aureus
  source 1..23: mol_type = other DNA
caagcagcagctgaagaaacagg

SEQ ID NO: 6 - DNA, 30 bp, Staphylococcus aureus
  source 1..30: mol_type = other DNA
tttgaatttatctgcactgttataaattgg

SEQ ID NO: 7 - DNA, 25 bp, Staphylococcus aureus
  source 1..25: mol_type = other DNA
actgaagaagattataaagctgaaa

SEQ ID NO: 8 - DNA, 22 bp, Staphylococcus aureus
  source 1..22: mol_type = other DNA
ttagtttttacgttttctaggt